(19) United States
(12) Patent Application Publication
(10) Pub. No.: US 2022/0089670 A1
(43) Pub. Date: Mar. 24, 2022

(54) Gene Therapy for BEST1 Dominant Mutations

(71) Applicants: UNIVERSITY OF ROCHESTER, Rochester, NY (US); THE TRUSTEES OF COLUMBIA UNIVERSITY IN THE CITY OF NEW YORK, New York, NY (US)

(72) Inventors: Tingting Yang, Rochester, NY (US); Yu Zhang, Rochester, NY (US); Stephen Tsang, New York, NY (US)

(21) Appl. No.: 17/415,858

(22) PCT Filed: Dec. 27, 2019
(86) PCT No.: PCT/US19/68690

§ 371 Date: Jun. 18, 2021

Related U.S. Application Data
(60) Provisional application No. 62/785,739, filed on Dec. 28, 2018.
(60) Provisional application No. 62/833,069, filed on Apr. 12, 2019.

(51) Int. Cl. C07K 14/705; A61K 48/00; C12N 5/0793; A61P 27/02

(52) U.S. Cl. CPC C07K 14/705; A61K 48/005; C12N 5/062; C12N 2510/00; C12N 2750/14143; A61K 48/0075; A61P 27/02

(57) ABSTRACT

The present invention provides compositions and methods for treatment of bestrophinopathies. In certain embodiments, the invention treats, improves retinal function, and prevents progression of disorder in a subject with retinal degenerative disorder. The present invention comprises administration of a composition comprising wild-type BEST1 gene to subjects with a BEST1 mutation, in need of improved retinal function.

### CROSS-REFERENCE TO RELATED APPLICATIONS

The present application claims priority to U.S. Provisional Patent Application Ser. No. 62/785,739, filed Dec. 28, 2018, and to U.S. Provisional Patent Application No. 62/833,069, filed Apr. 12, 2019, each of which is incorporated by reference herein in its entirety.

### STATEMENT REGARDING FEDERALLY SPONSORED RESEARCH OR DEVELOPMENT

This invention was made with government support under EY025290 and GM127652 awarded by National Institutes of Health. The government has certain rights in the invention.

### BACKGROUND OF THE INVENTION

Genetic mutation of the human BEST1 gene causes bestrophinopathies, which consist of a spectrum of retinal degeneration disorders including Best vitelliform macular dystrophy (BVMD) (Marquardt et al., 1998, Human Molecular Genetics, 7(9):1517-1525; Petrukhin et al., 1998, Nature Genetics 19(3):241-247), autosomal recessive bestrophinopathy (ARB)(Burgess et al., 2008, American Journal of Human Genetics, 82(1): 19-31), adult-onset vitelliform dystrophy (AVMD) (Allikmets R. et al., 1999, Human Genetics, 104(6):449-453; Kramer et al., 2000, European Journal of Human Genetics, 8(4):286-292), autosomal dominant vitreoretinochoroidopathy (ADVIRC) (Yardley et al., 2004, Investigative Ophthalmology & Visual Science, 45(10):3683-3689), and retinitis pigmentosa (RP) (Davidson et al., 2009, Proc. Natl. Acad. Sci, USA, 115(12):e2839-e2848). BVMD, featuring an early-onset and debilitating form of central macular degeneration, is the most common bestrophinopathy. Due to abnormalities in the fluid and/or electrolyte homeostasis between the RPE and photoreceptor outer segments (Yang et al., 2015, Molecular Therapy: The Journal of the American Society of Gene Therapy, 23(12):1805-1809), the disease leads to the formation of serous retinal detachment and lesions that resemble egg yolk, or vitelliform, while rod and cone photoreceptor function remains unaffected. All types of bestrophinopathies, except for ARB, result from autosomal dominant mutation of BEST1. Patients are susceptible to untreatable, progressive vision loss which significantly deteriorates life quality. Therefore, understanding the disease-causing mechanisms of BEST1 mutations and designing strategies to restore the damaged cellular function are critical for developing treatment of bestrophinopathies.

The protein encoded by BEST1 is a Cl⁻ channel named BESTROPHIN1 (BEST1), which is activated in response to intracellular Ca²⁺ and conducts Ca²⁺-dependent Cl⁻ current on the cell membrane of retinal pigment epithelium (RPE) (Li et al., 2017, Elife, 6: e29914; Marmorstein et al., 2000, Proc. Natl. Acad. Sci, USA, 97(23):12758-12763; Marquardt et al., 1998, Human Molecular Genetics, 7(9):1517-1525; Petrukhin et al., 1998, Nature Genetics 19(3):241-247). Consistently, Ca²⁺-dependent Cl⁻ current has been suggested to generate a critical visual response upon light exposure, namely light peak (LP) (Fujii et al., 1992, the American Journal of Physiology, 262:C374-383; Gallemore, R. P. et al., 1989, J Neuropsci, 9:1977-1984; Gallemore, R. P. et al., 1993, Journal of neurophysiology, 70:1669-1680), which is defective in almost all BEST1-mutated patients as shown by electrooculography (EOG) (Boon et al., 2009; Progress in retinal and eye research, 28:187-205; Marmorstein et al., 2009, Progress in retinal and eye research, 28: 206-226)). This BEST1-Cl⁻ current-LP correlation suggests gene compensation as a promising approach for curing bestrophinopathies. Indeed, it was reported that the impaired Cl⁻ current in RPE derived from an ARB patient bearing a BEST1 recessive mutation was rescuable by baculovirus (BV)-mediated supplementation of the Wild-type (WT) BEST1 gene (Li et al., 2017, Elife, 6: e29914). Moreover, a recent study in canine models demonstrated that the retinal abnormalities caused by recessive mutation of BEST1 can be corrected by adeno-associated virus (AAV)-mediated subretinal BEST1 gene augmentation (Guziewicz et al., 2018, Proc. Natl. Acad. Sci. USA, 115(12): E2839-E2848). However, the rescue efficacy of gene compensation for BEST1 dominant mutations is still unknown. This is a very important question because firstly, most of BEST1 mutations are dominant, and secondly, it will determine if disruption/suppression of the dominant mutant allele is necessary in therapeutic interventions. In principle, the excess of WT BEST1 could overwhelm the mutant BEST1 despite that the latter is dominant over the former at a 1:1 ratio. As canines do not have BEST1 dominant mutation genotypes while Best1 knock out mice do not show any retinal phenotype or Cl⁻ current abnormality (Marmorstein et al., 2006, Journal of Genetic Physiology, 127(5):577-589; Milenkovic et al., 2015, Proc. Natl. Acad. Sci USA, 112(20)-E2630-2639), patient-derived RPE provide a powerful model for testing the rescue of BEST1 dominant mutations.

Thus, there is a need in the art for curing bestrophinopathies by gene therapy, no matter if the causal BEST1 mutation is dominant or recessive. The present invention satisfies this unmet need.

### SUMMARY OF THE INVENTION

In one aspect, the present invention provides a method of treating a retinal degenerative disorder associated with a BEST1 dominant mutation in a subject. In one embodiment, the method comprises administering to a subject in need thereof an effective amount of a composition comprising a nucleic acid molecule encoding wild-type BEST1.

In one embodiment, the nucleic acid molecule encodes a polypeptide comprising an amino acid sequence comprising SEQ ID NO: 1. In one embodiment, the nucleic acid molecule comprises a nucleic acid sequence selected from the group consisting of SEQ ID NO: 2 and a nucleic acid sequence that is at least 90% homologous to SEQ ID NO: 2.

In one embodiment, the composition comprises a recombinant AAV promoter linked to the nucleic acid. In one embodiment, the recombinant AAV promoter is an AAV2 promoter.

In one embodiment, the composition comprises a recombinant AAV vector encoding BEST1. In one embodiment, the recombinant AAV vector is an AAV2 vector.

In one embodiment, the dominant mutation is selected from the group consisting of p.A10T, p.R218H, p.L234P, p.A243T, p.Q293K and p.D302A.

In one embodiment, the composition is administered via subretinal injection. In one embodiment, the composition is administered to a retinal pigment epithelial cells of the subject.

In one embodiment, the retinal degenerative disorder is a bestrophinopathy selected from the group consisting of: Best vitelliform macular dystrophy (BVMD), adult-onset vitelliform dystrophy (AVMD), autosomal dominant vitreoretinochoroidopathy (ADVIRC), and retinitis pigmentosa (RP).

In one embodiment, the subject is a mammal. In one embodiment, the mammal is a human.

In one aspect, the present invention provides a cell having an endogenous BEST1 dominant mutation comprising an exogenous nucleic acid molecule that encodes wild-type BEST1. In one embodiment, the exogenous nucleic acid molecule encodes polypeptide comprising an amino acid sequence comprising SEQ ID NO: 1. In one embodiment, the exogenous nucleic acid molecule comprises a nucleic acid sequence selected from the group consisting of SEQ ID NO: 2 and a nucleic acid sequence that is at least 90% homologous to SEQ ID NO: 2.

In one embodiment, the exogenous nucleic acid molecule comprises a recombinant AAV promoter linked to the wild-type BEST1. In one embodiment, the recombinant AAV promoter is an AAV2 vector.

In one embodiment, the exogenous nucleic acid molecule comprises a recombinant AAV vector encoding BEST1. In one embodiment, the recombinant AAV vector is an AAV2 vector. In one embodiment, the cell is retinal pigment epithelial cell.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A through FIG. 1G depict clinical phenotypes of six patients with BEST1 dominant mutations. FIG. 1A and FIG. 1C depict a fundus infrared reflectance image and Spectral Domain Optical Coherence Tomography (SDOCT) of the maculae from patient 1 (FIG. 1A) and patient 2 (FIG. 1B) and patient 3 (FIG. 1C), right and left eyes respectively. FIG. 1D depicts a fundus infrared image and SDOCT of patient 4 right eye. FIG. 1E depicts a fundus infrared reflectance image and SDOCT of a wildtype (WT) left eye. FIG. 1F and FIG. 1G depict a fundus infrared reflectance image and SDOCT of the maculae from patient 5 (FIG. 1F) and patient 6 (FIG. 1G), right and left eyes, respectively. Scale bar, 200 km.

FIG. 2A through FIG. 2G depicts disease-causing mechanisms of BEST1 mutations. FIG. 2A depicts bar chart showing population steady-state current densities at 100 mV for transiently expressed BEST1 WT and mutants in HEK293 cells at 1.2 μM [Ca²⁺]ᵢ; n=5-6 for each point. *P=4×10⁻⁵ (A10T), 7×10⁻⁴ (R218H), 2×10⁻³ (L234P), 3×10⁻² (A243T), 2×10⁻⁴ (Q293K), 2×10⁻³ (D302A), compared to WT, respectively, and ^{#}P=6×10⁻⁴ for A243T compared to untransfected cells, using two-tailed unpaired Student t test. FIG. 2B depicts a blot where WT or mutant BEST1-YFP-His was co-transfected with WT BEST1-CFP-Myc to HEK293 cells, and detected by immunoblotting directly in cell lysate (input) or after co-immunoprecipitation. The full-length blots are shown in FIG. 11. All error bars in this figure represent s.e.m. FIG. 1C depicts a ribbon diagram of two oppositely facing (144°) protomers of a BEST1 pentamer are shown with the extracellular side on the top. The side chains of critical residues are in red. FIG. 2D depicts the location of the patient mutations in relationship to the channel pore, as viewed from the side. A10 (red), Q293 (green) and D302 (blue) are colored differently. R218, L234 and A243 are colored by atoms. FIG. 2E depicts possible interactions of the mutated residues. Each monomeric unit is drawn by a different color and the mutated residues are colored in magenta. Coordination bonds and possible hydrogen bonds are illustrated by dotted black and yellow lines, respectively. FIG. 2F depicts the effect of replacing R218 with H. Possible conformations of H218 without steric hindrance are shown by magenta sticks. Each monomeric unit is drawn by a different color. Hydrogen bonds are illustrated by dotted yellow lines. FIG. 2G depicts the effect of replacing A243 and L234. Each monomeric unit is drawn by a different color and the mutated residues are colored in magenta. Possible van der Waals contact or steric hindrance are indicated by dotted red lines. A possible hydrogen bond is illustrated by a dotted yellow line.

FIG. 3 depicts the subcellular localization of WT and mutant BEST1 in iPSC-RPEs. Confocal images showing the co-staining of BEST1, Collagen IV and Hoechst in iPSC-RPEs derived from a WT donor or patients. Scale bar, 15 m.

FIG. 4A through FIG. 4G depicts surface Ca²⁺-dependent Cl⁻ currents in patient-derived iPSC-RPEs. FIG. 4A depicts Ca²⁺-dependent Cl⁻ currents measured by whole-cell patch clamp in patient-derived iPSC-RPEs bearing the mutation of A10T. Top, representative current traces recorded at 1.2 μM [Ca²⁺]ᵢ. Inset, voltage protocol used to elicit currents. Bottom, Ca²⁺-dependent current densities, n:=5-6 for each point, compared to WT (⋅). The WT plot was fitted to the Hill equation. FIG. 4B through FIG. 4F depict Ca²⁺-dependent Cl⁻ currents measured by whole-cell patch clamp in patient-derived iPSC-RPEs bearing the mutation of R218H (FIG. 4B), L234P (FIG. 4C), A243T (FIG. 4D), Q293K (FIG. 4E) and D302A (FIG. 4F), respectively. Top, representative current traces recorded at 1.2 LM [Ca²⁺]ᵢ. Bottom, Ca²⁺-dependent current densities, n=5-6 for each point, compared to WT (⋅). The WT, A234T and Q293K plots were fitted to the Hill equation. FIG. 4G depicts the comparison of current densities in iPSC-RPEs with WT or mutant BEST1 at 1.2 μM [Ca²⁺]ᵢ, n=5-6. Two clonal iPSC-RPEs from each patient. Black, W T. Gray, patient. All error bars in this figure represent s.e.m.

FIG. 5A through FIG. 5L depicts the rescue of patient-derived iPSC-RPEs by WT BEST1 supplementation. FIG. 5A depicts confocal images showing the expression of WT BEST1-GFP from BacMram virus in donor-derived iPSC-RPEs. FIG. 5B depicts representative current traces recorded from R218H iPSC-RPE (patient #2) supplemented with WT BEST1-GFP at 1.2 μM [Ca²⁺]ᵢ. FIG. 5C depicts current densities in R2181-1 iPSC-RPE supplemented with WIT BEST1-GFP (blue triangle) at 1.2 μM [Ca²⁺]ᵢ, compared to those from un-supplemented R218H (red triangle) and WT (⋅) iPSC-RPEs. n=5-6 for each point. *P=9×10⁻⁴ compared to WIT, using two-tailed unpaired Student t test. FIG. 5D depicts Ca²⁺-dependent current densities in R218H iPSC-RPE supplemented with WT BEST1-GFP (blue triangle) compared to those from WT (⋅) iPSC-RPE. Steady-state current density recorded at +100 mV plotted vs. free [Ca²⁺]ᵢ; n=5-6 for each point. The plots were fitted to the Hill equation. FIG. 5E depicts current densities at 1.2 μM [Ca²⁺]ᵢ in a second clone of R218H iPSC-RPE supplemented with different dosages of WT BEST1-GFP on BacMam viruses. n=5-6 for each point. FIG. 5F through FIG. 5J depicts Ca²⁺-dependent current densities in patient-derived iPSC-RPEs bearing the mutation of A10T (FIG. 5F), L234P (FIG. 5G), A243T (FIG. 5H), Q293K (FIG. 5I) and D302A (FIG. 5J), supplemented with WT BEST1-GFP (blue triangle), compared to those from WT (⋅) iPSC-RPE, n=5-6 for each point. The plots were fitted to the Hill equation. FIG. 5K depicts current densities at L2 M [Ca²⁺]ᵢ in the second clones of the five BEST1 dominant iPSC-RPEs and a clone of the recessive P274R iPSC-RPE supplemented with different dosages of WT BEST1-GFP from BacMam viruses on day 2. FIG. 5L depicts current densities at 1.2 μM [Ca²⁺]ᵢ in patient-derived iPSC-RPEs supplemented with WT BEST1 on AAV2 viruses. All error bars in this figure represent s.e.m.

FIG. 6A through FIG. 6F depicts fundus photographs from patients. FIG. 6A through FIG. 6F depicts fundus photographs from indicated patients, right and left eye, respectively, except for FIG. 6D, in which both photographs are from the right eye of the patient.

FIG. 7A through FIG. 7B depicts the lack of light rise in EOG profiles of patients #4 and #5. FIG. 7A through FIG. 7B depicts the EOG profiles of BEST1 p.A243T (FIG. 7A) and p.Q293K (FIG. 7B) patients (red) were compared to that of a BEST1 WT (black) person. Right and left eye, respectively. Scale bar, 2 μV/deg, 5 min.

FIG. 8A through FIG. 8F depicts channel activity of BEST1 mutants in HEK293 cells. FIG. 8A through FIG. 8F depicts Ca²⁺-dependent Cl⁻ currents at 1.2 μM [Ca²⁺]ᵢ in HEK293 cells transiently expressing indicated BEST1 mutants (red), compared to the WT (black), n=5-6 for each point. *P<0.05 compared to WT cells, using two-tailed unpaired Student t test. Controls are from the same set of data. All error bars in this figure represent s.e.m.

FIG. 9A through FIG. 9D depict protein expression in RPE cells. FIG. 9A depicts the expression of marker proteins in WT and patient-derived iPSC-RPEs. The expression of RPE-specific proteins BEST1, RPE65 and CRALBP were detected by immunoblotting. Two gels/blots were prepared from the same cell lysate of each iPSC-RPE to detect BEST1+CRALBP, and RPE65+β-Actin, respectively. Full-length blots are shown in FIG. 11. FIG. 9B depicts cell surface expression of BEST1 in iPSC-RPEs was detected by immunoblotting (FIG. 9B, top). Membrane extractions were generated from the same batch of cell pellets as in FIG. 9A. Quantitation of the levels of BEST1 in plasma membrane from 3 independent experiments is also depicted (FIG. 9B, bottom). Data were normalized to the loading control global BEST1 and then compared to WT. *P<0.05 compared to WT cells, using two-tailed unpaired Student t test. All error bars in this figure represent s.e.m. FIG. 9C depicts a blot where Baculovirus supplemented exogenous BEST1-GFP (WT) and endogenous BEST1 (WT/mutant) in whole cell lysate were detected by immunoblotting. FIG. 9D depicts the expression of the loading control β-Actin from the same set of samples FIG. 9C, as detected by immunoblotting.

FIG. 10A through FIG. 10F depicts Ca²⁺-dependent Cl⁻ currents in patient-derived iPSC-RPEs. FIG. 10A through FIG. 10F depicts Ca²⁺-dependent Cl-currents at 1.2 μM [Ca²⁺]ᵢ in indicated patient-derived iPSC-RPEs supplemented with WT BEST1-GFP(blue ▴), compared to un-supplemented mutant (red ▴), and WT (●) iPSC-RPEs. n=5-6 for each point. *P<0.05 compared to WT cells, using two-tailed unpaired Student t test. Controls are from the same set of data. All error bars in this figure represent s.e.m.

FIG. 11 depicts the full-length blots of those shown in FIG. 2B and FIG. 9A.

FIG. 12 depicts a table with patient information.

### DETAILED DESCRIPTION

The present invention provides compositions and methods for treatment of bestrophinopathies in a subject. The present invention relates to strategies of delivering the wild-type BEST1 gene to subjects in need of improved retinal function. The compositions and methods of the present invention improve visual function and prevent disease progression in a subject in need thereof.

In one embodiment, the present invention is useful for treating a subject with a retinal degenerative disorder. The present invention is based upon the findings that delivery of an adeno-associated viral vector encoding wild-type BEST1 drastically improves retinal function. Thus, the present invention provides non-invasive compositions and methods to treat bestrophinopathies, including, but not limited to, best vitelliform macular dystrophy (BVMD), adult-onset vitelliform dystrophy (AVMD), autosomal dominant vitreoretinochoroidopathy (ADVIRC), and retinitis pigmentosa (RP).

In one embodiment, the present invention is useful for treating a subject with bestrophinopathies, characterized by a loss of function dominant mutation in BEST1 gene. The present invention improves retinal function and prevents progression of disorder in afflicted subjects. In certain embodiments, the method rescues a loss-of function BEST1 mutation, including an autosominal dominant or negative mutation. In one embodiment, the dominant mutation is selected from the group consisting of p.R218H, p.A243T, p.A10T, p.L234P, p.Q293K and p.D302A. In one embodiment, the present invention is useful in restoration of Ca²⁺-dependent Cl⁻ current on the cell membrane. In certain embodiments, the method does not require the disruption or suppression of the mutant allele of the subject.

In one embodiment, the present invention provides a composition that increases the expression of wild-type BEST1 in a subject. For example, in one embodiment, the composition comprises a peptide comprising wild-type BEST1 protein, or biologically functional fragment thereof. In one embodiment the composition comprises a nucleic acid molecule encoding wild-type BEST1 or a biologically functional fragment thereof. In one embodiment, the composition comprises a nucleic acid molecule encoding a polypeptide comprising an amino acid sequence comprising SEQ ID NO: 1. In another embodiment, the composition comprises a nucleic acid molecule comprising a nucleic acid sequence that is at least 90%, at least 95%, at least 98%, or at least 99% homologous to SEQ ID NO: 2. In one embodiment, the composition comprises a viral vector comprising a nucleic acid sequence encoding BEST1.

In one embodiment, the present invention provides a method for improving retinal function comprising administering an effective amount of a composition which increases wild-type BEST1 expression in a subject. For example, in one embodiment, the composition comprises a peptide comprising wild-type BEST1 protein, or biologically functional fragment thereof. In one embodiment, the method comprises administering to a subject a composition comprising a nucleic acid molecule encoding wild-type BEST1 or a biologically functional fragment thereof. In another embodiment, the composition comprises a nucleic acid molecule encoding a polypeptide comprising an amino acid sequence comprising SEQ ID NO: 1. In another embodiment, the composition comprises the nucleic acid molecule comprising a nucleic acid sequence selected from the group consisting of SEQ ID NO: 2 and a nucleic acid sequence that is at least 90% homologous to SEQ ID NO: 2.

In one embodiment, the method comprises administering a composition comprising a viral vector comprising the wild-type BEST1 gene to a subject in need of improved retinal function. In one embodiment, the method comprises administering to a subject a composition comprising a nucleic acid molecule encoding wild-type BEST1. In one embodiment, the composition comprises a nucleic acid molecule encoding a polypeptide comprising an amino acid sequence comprising SEQ ID NO: 1. In one embodiment, the composition comprises the nucleic acid molecule comprising a nucleic acid sequence selected from the group consisting of SEQ ID NO: 2 and a nucleic acid sequence that is at least 90% homologous to SEQ ID NO: 2.

In one embodiment, the present invention provides a cell having an endogenous BEST1 dominant mutation comprising an exogenous nucleic acid molecule that encodes wild-type BEST1 or a biologically functional fragment thereof. For example, in one embodiment, the exogenous nucleic acid molecule encodes a polypeptide comprising an amino acid sequence comprising SEQ ID NO: 1. In one embodiment, the exogenous nucleic acid molecule comprises a nucleic acid sequence selected from the group consisting of SEQ ID NO: 2 and a nucleic acid sequence that is at least 90%, at least 95%, at least 98%, or at least 99% homologous to SEQ ID NO: 2.

In certain embodiments, the composition described above is administered to the subject by subretinal injection. In certain embodiments, the composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired. In certain embodiments, the route of administration is subretinal injection or intravitreal injection.

In one embodiment, the method comprises a single injection of a composition comprising a viral vector comprising the wild-type BEST1 gene. As described herein, the present invention is partly based upon the discovery that a single delivery of a viral vector comprising the wild-type BEST1 gene returned retinal function in both dominant and recessive cases. In certain embodiments, the method does not require the disruption or suppression of the mutant allele of the subject.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

As used herein, “treating a disease or disorder” means reducing the frequency with which a symptom of the disease or disorder is experienced by a patient.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. A vector may be a DNA or RNA vector. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention provides compositions and methods for improving retinal function. In one embodiment, the present invention provides for a treatment of a bestrophinopathies. The bestrophinopathies may be any form of bestrophinopathies including inherited bestrophinopathies. For example, in one embodiment, the present invention provides compositions and methods for treating bestrophinopathies resulting from autosomal dominant mutation of BEST1. The present invention provides for the ability to improve retinal function in any subject in need of improved retinal function. For example, in one embodiment, the present invention improves retinal function in subjects with progressive vision loss.

The present invention is partly based upon the findings that subretinal delivery of a viral vector comprising the wild-type BEST1 gene drastically improves retinal function and prevents progression of disorder in afflicted subjects. Thus, the compositions and methods described herein are useful in that they provide an easy and efficient treatment of retinal degenerative disorders, including bestrophinopathies.

### Compositions

The present invention provides a composition that increases the expression of wild-type BEST1, or biologically functional fragment thereof, in the retina. For example, in one embodiment the composition comprises a peptide comprising wild-type BEST1 protein, a variant thereof, or a biologically functional fragment thereof. In one embodiment the composition comprises a nucleic acid molecule encoding wild-type BEST1, a variant thereof, or a biologically functional fragment thereof. In one embodiment, the composition comprises a nucleic acid molecule encoding a polypeptide comprising an amino acid sequence comprising SEQ ID NO: 1. In one embodiment, the composition comprises the nucleic acid molecule comprising a nucleic acid sequence selected from the group consisting of SEQ ID NO: 2 and a nucleic acid sequence that is at least 90%, at least 95%, at least 98%, or at least 99% homologous to SEQ ID NO: 2. In one embodiment, the composition comprises a viral vector which includes a nucleic acid sequence encoding BEST1, a variant thereof, or a biologically functional fragment thereof.

In one embodiment, the composition comprises an isolated nucleic acid comprising a sequence encoding wild-type BEST1, a variant thereof, or a biologically functional fragment thereof. The isolated nucleic acid sequence encoding wild-type BEST1 can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

In certain embodiments, the composition increases the expression of a biologically functional fragment of wild-type BEST1. For example, in one embodiment, the composition comprises an isolated nucleic acid sequence encoding a biologically functional fragment of wild-type BEST1. As would be understood in the art, a biologically functional fragment is a portion or portions of a full-length sequence that retain the biological function of the full-length sequence. Thus, a biologically functional fragment of wild-type BEST1 comprises a peptide that retains the function of full length wild-type BEST1.

Further, the invention encompasses an isolated nucleic acid encoding a peptide having substantial homology to the peptides disclosed herein. Preferably, the nucleotide sequence of an isolated nucleic acid encoding a peptide of the invention is “substantially homologous”, that is, is about 60% homologous, about 70% homologous, about 80% homologous, about 90% homologous, about 91% homologous, about 92% homologous, about 93% homologous, about 94% homologous, about 95% homologous, about 96% homologous, about 97% homologous, about 98% homologous, or about 99% homologous to a nucleotide sequence of an isolated nucleic acid encoding a peptide of the invention.

In one embodiment, the composition of the invention comprises RNA encoding wild-type BEST1, a variant thereof, or a biologically-functional fragment thereof. For example, in one embodiment, the composition comprises in vitro transcribed (IVT) RNA encoding wild-type BEST1 protein, a variant thereof, or biologically-functional fragment thereof. In one embodiment, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is one or more wild-type BEST1 proteins. In one embodiment, the DNA to be used for PCR contains an open reading frame. The DNA can be from a naturally occurring DNA sequence from the genome of an organism. In one embodiment, the DNA is a full-length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the composition of the present invention comprises a modified nucleic acid encoding wild-type BEST1 protein described herein. For example, in one embodiment, the composition comprises a nucleoside-modified RNA. In one embodiment, the composition comprises a nucleoside-modified mRNA. Nucleoside-modified mRNA have particular advantages over non-modified mRNA, including for example, increased stability, low immunogenicity, and enhanced translation.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Vectors

The present invention also includes a vector in which the isolated nucleic acid of the present invention is inserted. The art is replete with suitable vectors that are useful in the present invention.

In brief summary, the expression of natural or synthetic nucleic acids encoding wild-type BEST1 is typically achieved by operably linking a nucleic acid encoding the wild-type BEST1 or portions thereof to a promoter and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In a preferred embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

In one embodiment, the wild-type BEST1 encoding sequence is contained within an AAV vector. More than 30 naturally occurring serotypes of AAV are available. Many natural variants in the AAV capsid exist, allowing identification and use of an AAV with properties specifically suited for retina. AAV viruses may be engineered using conventional molecular biology techniques, making it possible to optimize these particles for cell specific delivery of wild-type BEST1 nucleic acid sequences, for minimizing immunogenicity, for tuning stability and particle lifetime, for efficient degradation, for accurate delivery to the nucleus, etc.

Thus, wild-type BEST1 overexpression can be achieved in the retina by delivering a recombinantly engineered AAV or artificial AAV that contains sequences encoding wild-type BEST1. The use of AAVs is a common mode of exogenous delivery of DNA as it is relatively non-toxic, provides efficient gene transfer, and can be easily optimized for specific purposes. Among the serotypes of AAVs isolated from human or non-human primates (NHP) and well characterized, human serotype 2 is the first AAV that was developed as a gene transfer vector; it has been widely used for efficient gene transfer experiments in different target tissues and animal models. Clinical trials of the experimental application of AAV2 based vectors to some human disease models are in progress, and include therapies for diseases such as for example, cystic fibrosis and hemophilia B. Other useful AAV serotypes include AAV1, AAV3, AAV4, AAV5, AAV6, AAV7, AAV8 and AAV9.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. Thus, exemplary AAVs, or artificial AAVs, suitable for expression of wild-type BEST1, include AAV2/8 (see U.S. Pat. No. 7,282,199), AAV2/5 (available from the National Institutes of Health), AAV2/9 (International Patent Publication No. WO2005/033321), AAV2/6 (U.S. Pat. No. 6,156,303), and AAVrh8 (International Patent Publication No. WO2003/042397), among others.

In one embodiment, the vectors useful in the compositions and methods described herein contain, at a minimum, sequences encoding a selected AAV serotype capsid, e.g., an AAV2 capsid, or a fragment thereof. In another embodiment, useful vectors contain, at a minimum, sequences encoding a selected AAV serotype rep protein, e.g., AAV2 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV2 origin. Alternatively, vectors may be used in which the rep sequences are from an AAV serotype which differs from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 described in U.S. Pat. No. 7,282,199.

A suitable recombinant adeno-associated virus (AAV) is generated by culturing a host cell which contains a nucleic acid sequence encoding an adeno-associated virus (AAV) serotype capsid protein, or fragment thereof, as defined herein; a functional rep gene; a minigene composed of, at a minimum, AAV inverted terminal repeats (ITRs) and a wild-type BEST1 nucleic acid sequence, or biologically functional fragment thereof; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

Most suitably, such a stable host cell will contain the required component(s) under the control of an inducible promoter. However, the required component(s) may be under the control of a constitutive promoter. Examples of suitable inducible and constitutive promoters are provided elsewhere herein, and are well known in the art. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing the rAAV of the invention may be delivered to the packaging host cell in the form of any genetic element which transfers the sequences carried thereon. The selected genetic element may be delivered using any suitable method, including those described herein and any others available in the art. The methods used to construct any embodiment of this invention are known to those with skill in nucleic acid manipulation and include genetic engineering, recombinant engineering, and synthetic techniques (see, e.g., Sambrook et al, Molecular Cloning: A Laboratory Manual, Cold Spring Harbor Press, Cold Spring Harbor, N.Y). Similarly, methods of generating rAAV virions are well known and the selection of a suitable method is not a limitation on the present invention (see, e.g., K. Fisher et al, 1993 J. Virol., 70:520-532 and U.S. Pat. No. 5,478,745, among others).

Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9 or other known or as yet unknown AAV serotypes. These ITRs or other AAV components may be readily isolated from an AAV serotype using techniques available to those of skill in the art. Such an AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

In addition to the major elements identified above for the minigene, the AAV vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. In one embodiment, the vector of the invention comprises a tissue-specific promoter to drive expression of wild-type BEST1 in one or more specific types of cells. In one embodiment, the vector of the invention comprises a tissue-specific promoter to drive expression of wild-type BEST1 specifically in retina.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector. For example, in one embodiment, the vector of the invention comprises a retina-specific enhancer to enhance wild-type BEST1 expression specifically in retina.

In order to assess the expression of wild-type BEST1, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In one embodiment, the composition comprises a naked isolated nucleic acid encoding wild-type BEST1, or a biologically functional fragment thereof, wherein the isolated nucleic acid is essentially free from transfection-facilitating proteins, viral particles, liposomal formulations and the like (see, for example U.S. Pat. No. 5,580,859). It is well known in the art that the use of naked isolated nucleic acid structures, including for example naked DNA, works well with inducing expression in retina. As such, the present invention encompasses the use of such compositions for local delivery to the retina and for systemic administration (Wu et al., 2005, Gene Ther, 12(6): 477-486).

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### Peptide/Polypeptide

In one embodiment, the composition of the present invention comprises a peptide comprising wild-type BEST1 protein, a variant thereof, or a biologically functional fragment thereof. The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The invention should also be construed to include any form of a peptide having substantial homology to the peptides disclosed herein. Preferably, a peptide which is “substantially homologous” is about 60% homologous, about 70% homologous, about 80% homologous, about 90% homologous, about 91% homologous, about 92% homologous, about 93% homologous, about 94% homologous, about 95% homologous, about 96% homologous, about 97% homologous, about 98% homologous, or about 99% homologous to amino acid sequence of the peptides disclosed herein.

The peptide may alternatively be made by recombinant means or by cleavage from a longer polypeptide. The composition of a peptide may be confirmed by amino acid analysis or sequencing.

The variants of the polypeptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two polypeptides is determined by comparing the amino acid sequence and its conserved amino acid substitutes of one polypeptide to a sequence of a second polypeptide. Variants are defined to include polypeptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence and/or the ability to bind to ubiquitin or to a ubiquitylated protein. The present invention includes amino acid sequences that are at least 60%, 65%, 70%, 72%, 74%, 76%, 78%, 80%, 90%, or 95% similar or identical to the original amino acid sequence. The degree of identity between two polypeptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The polypeptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

The term “functionally equivalent” as used herein refers to a polypeptide according to the invention that preferably retains at least one biological function or activity of the specific amino acid sequence of wild-type BEST1.

A peptide or protein of the invention may be conjugated with other molecules, such as proteins, to prepare fusion proteins. This may be accomplished, for example, by the synthesis of N-terminal or C-terminal fusion proteins provided that the resulting fusion protein retains the functionality of the wild-type BEST1 comprising peptide.

A peptide or protein of the invention may be phosphorylated using conventional methods such as the method described in Reedijk et al. (The EMBO Journal 11(4):1365, 1992).

Cyclic derivatives of the peptides or chimeric proteins of the invention are also part of the present invention. Cyclization may allow the peptide or chimeric protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

(a) Tags

In a particular embodiment of the invention, the polypeptide of the invention further comprises the amino acid sequence of a tag. The tag includes but is not limited to: polyhistidine tags (His-tags) (for example H6 and H10, etc.) or other tags for use in IMAC systems, for example, Ni²⁺ affinity columns, etc., GST fusions, MBP fusions, streptavidine-tags, the BSP biotinylation target sequence of the bacterial enzyme BIRA and tag epitopes that are directed by antibodies (for example c-myc tags, FLAG-tags, among others). As will be observed by a person skilled in the art, the tag peptide can be used for purification, inspection, selection and/or visualization of the fusion protein of the invention. In a particular embodiment of the invention, the tag is a detection tag and/or a purification tag. It will be appreciated that the tag sequence will not interfere in the function of the protein of the invention.

(b) Leader and Secretory Sequences

Accordingly, the polypeptides of the invention can be fused to another polypeptide or tag, such as a leader or secretory sequence or a sequence which is employed for purification or for detection. In a particular embodiment, the polypeptide of the invention comprises the glutathione-S-transferase protein tag which provides the basis for rapid high-affinity purification of the polypeptide of the invention. Indeed, this GST-fusion protein can then be purified from cells via its high affinity for glutathione. Agarose beads can be coupled to glutathione, and such glutathione-agarose beads bind GST-proteins. Thus, in a particular embodiment of the invention, the polypeptide of the invention is bound to a solid support. In a preferred embodiment, if the polypeptide of the invention comprises a GST moiety, the polypeptide is coupled to a glutathione-modified support. In a particular case, the glutathione modified support is a glutathione-agarose bead. Additionally, a sequence encoding a protease cleavage site can be included between the affinity tag and the polypeptide sequence, thus permitting the removal of the binding tag after incubation with this specific enzyme and thus facilitating the purification of the corresponding protein of interest.

(c) Targeting Sequences

The invention also relates to peptides comprising wild-type BEST1 fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e. are heterologous).

A target protein is a protein that is selected for degradation and for example may be a protein that is mutated or over expressed in a disease or condition. In another embodiment of the invention, a target protein is a protein that is abnormally degraded and for example may be a protein that is mutated or underexpressed in a disease or condition. The targeting domain can be a membrane spanning domain, a membrane binding domain, or a sequence directing the protein to associate with for example vesicles or with the nucleus. The targeting domain can target a peptide to a particular cell type or tissue. For example, the targeting domain can be a cell surface ligand or an antibody against cell surface antigens of a target tissue (e.g. retina tissue). A targeting domain may target the peptide of the invention to a cellular component.

(d) Intracellular Targeting

Combined with certain formulations, such peptides can be effective intracellular agents. However, in order to increase the efficacy of such peptides, the peptide of the invention can be provided a fusion peptide along with a second peptide which promotes “transcytosis”, e.g., uptake of the peptide by epithelial cells. To illustrate, the peptide of the present invention can be provided as part of a fusion polypeptide with all or a fragment of the N-terminal domain of the HIV protein Tat, e.g., residues 1-72 of Tat or a smaller fragment thereof which can promote transcytosis. In other embodiments, the peptide can be provided a fusion polypeptide with all or a portion of the antenopedia III protein.

To further illustrate, the peptide of the invention can be provided as a chimeric peptide which includes a heterologous peptide sequence (“internalizing peptide”) which drives the translocation of an extracellular form of the peptide across a cell membrane in order to facilitate intracellular localization of the peptide. In this regard, the peptide is one which is active intracellularly. The internalizing peptide, by itself, is capable of crossing a cellular membrane by, e.g., transcytosis, at a relatively high rate. The internalizing peptide is conjugated, e.g., as a fusion protein, to a peptide comprising wild-type BEST1. The resulting chimeric peptide is transported into cells at a higher rate relative to the peptide alone to thereby provide a means for enhancing its introduction into cells to which it is applied.

(e) Peptide Mimetics

In other embodiments, the subject compositions are peptidomimetics of the peptide of the invention. Peptidomimetics are compounds based on, or derived from, peptides and proteins. The peptidomimetics of the present invention typically can be obtained by structural modification of a known sequence using unnatural amino acids, conformational restraints, isosteric replacement, and the like. The subject peptidomimetics constitute the continuum of structural space between peptides and nonpeptide synthetic structures; peptidomimetics may be useful, therefore, in delineating pharmacophores and in helping to translate peptides into nonpeptide compounds with the activity of the parent peptides.

Moreover, as is apparent from the present disclosure, mimotopes of the subject peptides can be provided. Such peptidomimetics can have such attributes as being non-hydrolysable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p 123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), 3-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modifed (Roark et al. in Peptides: Chemistry and Biology, G. R. Marshall ed., ESCOM Publisher: Leiden, Netherlands, 1988, p 134). Also, see generally, Session III: Analytic and synthetic methods, in in Peptides: Chemistry and Biology, G. R. Marshall ed., ESCOM Publisher: Leiden, Netherlands, 1988)

In addition to a variety of side chain replacements which can be carried out to generate the peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of binding to the peptide of the invention. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modelling, the predicted mimetope structures can then be produced by, for example, chemical synthesis, recombinant DNA technology, or by isolating a mimetope from a natural source (e.g., plants, animals, bacteria and fungi).

A peptide of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.) By way of example, a protein or chimeric protein may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or chimeric protein of the invention conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain the wild-type BEST1 comprising peptide or chimeric protein fused to the selected protein or marker protein as described herein. Examples of proteins which may be used to prepare fusion proteins include immunoglobulins, glutathione-S-transferase (GST), hemagglutinin (HA), and truncated myc.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (See Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

### Modified Cell

The present invention includes a cell having an endogenous BEST1 mutation comprising an exogenous nucleic acid molecule that encodes wild-type BEST1, a variant thereof, or a biologically functional fragment thereof. In one embodiment, the exogenous nucleic acid molecule encodes polypeptide comprising an amino acid sequence comprising SEQ ID NO: 1. In one embodiment, the exogenous nucleic acid molecule comprises a nucleic acid sequence selected from the group consisting of SEQ ID NO: 2 and a nucleic acid sequence that is at least 90% homologous to SEQ ID NO: 2.

In one embodiment, the cell is genetically modified to express a protein and/or nucleic acid of the invention. In certain embodiments, genetically modified cell is autologous to a subject being treated with the composition of the invention. Alternatively, the cells can be allogeneic, syngeneic, or xenogeneic with respect to the subject. In certain embodiment, the cell is able to secrete or release the expressed protein into extracellular space in order to deliver the peptide to one or more other cells.

The genetically modified cell may be modified in vivo or ex vivo, using techniques standard in the art. Genetic modification of the cell may be carried out using an expression vector or using a naked isolated nucleic acid construct.

In one embodiment, the cell is obtained and modified ex vivo, using an isolated nucleic acid encoding one or more proteins described herein. In one embodiment, the cell is obtained from a subject, genetically modified to express the protein and/or nucleic acid, and is re-administered to the subject. In certain embodiments, the cell is expanded ex vivo or in vitro to produce a population of cells, wherein at least a portion of the population is administered to a subject in need.

In one embodiment, the cell is genetically modified to stably express the protein. In another embodiment, the cell is genetically modified to transiently express the protein.

### Therapeutic Methods

The present invention encompasses a method to treat bestrophinopathy in a subject diagnosed with a bestrophinopathy or in a subject at risk for developing a bestrophinopathy. In certain embodiments, the bestophinophaty of the subject is associated with a loss-of-function dominant mutation. In one embodiment, the dominant mutation is selected from the group consisting of p.R218H, p.A243T, p.A10T, p.L234P, p.Q293K and p.D302A. The method improves retinal strength and retinal function in those in need. Further, the method improves quality of life and prevent disease progression in a patient with a bestrophinopathy. In one embodiment, the method of the present invention comprises administering to a subject, a composition comprising the wild-type BEST1 gene, a variant thereof, or a biologically functional fragment thereof. In one embodiment, the method of the present invention comprises administering to a subject, a composition comprising a nucleic acid sequence encoding wild-type BEST1, a variant thereof, or a biologically functional fragment thereof. In another embodiment, the method comprises inducing the expression of wild-type BEST1, a variant thereof, or a biologically functional fragment thereof specifically in the retina of the subject.

The method of the present invention is used to treat any type of bestrophinopathy in a subject. A bestrophinopathy is a retinal dystrophy, characterized by central visual loss. In one embodiment, the method of the present invention is used to treat a spectrum of retinal degenerative disorders. Exemplary retinal degeneration disorders that can be treated by way of the presently described methods includes, but is not limited to, best vitelliform macular dystrophy (BVMD), autosomal recessive bestrophinopathy (ARB), adult-onset vitelliform dystrophy (AVMD), autosomal dominant vitreoretinochoroidopathy (ADVIRC), and retinitis pigmentosa (RP).

Compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials. When “an effective amount”, or “therapeutic amount” is indicated, the precise amount of the compositions of the present invention to be administered can be determined by a physician with consideration of individual differences in age, weight, disease progression, and condition of the patient (subject). The optimal dosage and treatment regime for a particular patient can readily be determined by one skilled in the art of medicine by monitoring the subject for signs of disease and adjusting the treatment accordingly.

The administration of the subject compositions may be carried out in any convenient manner, including by aerosol inhalation, injection, ingestion, transfusion, implantation or transplantation. The compositions described herein may be administered to a subject subcutaneously, intradermally, intratumorally, intranodally, intramedullary, intramuscularly, by intravenous (i.v.) injection, or intraperitoneally. In one embodiment, the method of the invention comprises a systemic administration of a composition comprising wild-type BEST1.

In certain embodiments, the composition described above is administered to the subject by subretinal injection. In other embodiments, the composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired. In one embodiment, the route of administration is subretinal injection or intravitreal injection. The present invention is partly based upon the discovery that subretinal administration of a vector comprising wild-type BEST1 improved visual function and prevent disease progression.

The certain embodiments of the present invention, the composition, as described herein, are administered to a subject in conjunction with (e.g. before, simultaneously, or following) any number of relevant treatment modalities.

### Dosage and Formulation (Compositions)

The present invention envisions treating a disease, for example, bestrophinopathy and the like, in a subject by the administration of therapeutic agent, e.g. a composition comprising a viral vector comprising the wild-type BEST1 gene, a variant thereof, or a biologically functional fragment thereof.

Administration of the composition or modified cell in accordance with the present invention may be continuous or intermittent, depending, for example, upon the recipient's physiological condition, whether the purpose of the administration is therapeutic or prophylactic, and other factors known to skilled practitioners. The administration of the agents or modified cell of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent or modified cell may be directly injected into the muscle. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The expression vectors, transduced cells, polynucleotides and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. In one embodiment, the composition described above is administered to the subject by subretinal injection. In other embodiments, the composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired. In another embodiments, route of administration is subretinal injection or intravitreal injection.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Gene Therapy Administration

One skilled in the art recognizes that different methods of delivery may be utilized to administer a vector into a cell. Examples include: (1) methods utilizing physical means, such as electroporation (electricity), a gene gun (physical force) or applying large volumes of a liquid (pressure); and (2) methods wherein the vector is complexed to another entity, such as a liposome, aggregated protein or transporter molecule.

Furthermore, the actual dose and schedule can vary depending on whether the compositions are administered in combination with other compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

Cells containing the therapeutic agent may also contain a suicide gene i.e., a gene which encodes a product that can be used to destroy the cell. In many gene therapy situations, it is desirable to be able to express a gene for therapeutic purposes in a host, cell but also to have the capacity to destroy the host cell at will. The therapeutic agent can be linked to a suicide gene, whose expression is not activated in the absence of an activator compound. When death of the cell in which both the agent and the suicide gene have been introduced is desired, the activator compound is administered to the cell thereby activating expression of the suicide gene and killing the cell. Examples of suicide gene/prodrug combinations which may be used are herpes simplex virus-thymidine kinase (HSV-tk) and ganciclovir, acyclovir; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Investigation and Restoration of BEST1 Activity in Patient-Derived RPEs with Dominant Mutations

Six BEST1 dominant disease-causing mutations (A10T, R218H, L234P, A243T, Q293K and D302A) derived from BVMD patients were examined in an interdisciplinary platform, including whole-cell patch clamp with patient-derived iPSC-RPEs and HEK293 cells expressing the mutant channels, immunodetection of endogenous BEST1 in iPSC-RPEs, structural analyses with human homology models, and virus-mediated BEST1 gene supplementation. Collectively, these results illustrated the physiological influence of these six dominant mutations on RPE surface Ca²⁺-dependent Cl⁻ current and the BEST1 channel function, provided structural insights into their disease-causing mechanisms, and demonstrated the rescue of BEST1 dominant mutations in iPSC-RPE via gene supplementation. Notably, the diminished Ca²⁺-dependent Cl⁻ currents in the R218H, L234P and A243T patient-derived iPSC-RPEs are in accord with the deficient Ca²⁺-stimulated Cl⁻ secretion shown by Cl⁻ sensitive fluorescent dyes in these cells (Moshfegh, Y. et al., 2016, Human molecular genetics, 25:2672-2680).

Previously, it was reported that the impaired Ca²⁺-dependent Cl⁻ current in iPSC-RPE derived from an ARB patient bearing a BEST1 recessive mutation (P274R) was rescuable by BV-mediated supplementation of WT BEST1 (Li, Y. et al., 2017, Elife 6). Here, it was shown that the same strategy can be generally utilized for restoring Ca²⁺-dependent Cl⁻ current impaired by BEST1 dominant mutations, most of which are loss-of-function, and can be further classified into null (e.g. A10T, R218H and D302A), and partial deficiency with unaffected (e.g. L234P and A243T) or shifted (e.g. Q293K) Ca²⁺-sensitivity.

The retina has been the frontier of translational gene therapy in the past 20 years. Recently, the first gene therapy drug, an AAV-based vector carrying a correct copy of the RPE65 gene, was approved by FDA for treating retinal degenerative Leber congenital amaurosis type 2 (LCA2), which is caused by recessive mutations in RPE65 (Bainbridge, J. W. et al., 2008, The New England journal of medicine, 358:2231-2239; Bennett, J. et al., 2016, Lancet, 388:661-672; Jacobson, S. G. et al., 2012, Arch Ophthalmol, 130:9-24; Russell et al., 2017, Lancet, 849-860; Testa, F. et al., 2013, Ophthalmology, 120:1283-1291). As another inherited retinal disorder clearly linked to the mutation of a single gene, Best disease represents an attractive target of gene therapy. However, since the vast majority of known BEST1 mutations are autosomal dominant, it remains a critical question whether the dominant mutant allele should be purposely suppressed during gene therapy. These results showed that virus-mediated WT BEST1 gene supplementation restores the deficient Ca²⁺-dependent Cl⁻ currents in patient-derived iPSC-RPEs with the same dose- and time-dependent efficacy regardless of the mutation type (dominant vs. recessive) or deficiency level (complete vs. partial), providing the first line of evidence that most of disease-causing BEST1 mutations are rescuable by WT gene compensation without the need of disrupting/suppressing the mutant allele(s). Moreover, as AAV-mediated subretinal BEST1 gene augmentation therapy has succeeded in reversing clinically detectable subretinal lesions and diffuse microdetachments in canine BEST1 recessive mutation models (Guziewicz, K. E. et al., Proc Natl Acad Sci USA, 115:E2839-E2848), it is examined herein whether gene supplementation can be applied to treat patients with either dominant or recessive BEST1 mutations as long as the outcome is loss of BEST1 function.

Several gain-of-function mutations have been recently identified, which significantly enhance BEST1 channel activity (Ji, C. et al., Commun Biol). Although the pathological basis of elevated BEST1 activity remains unclear, it is speculated that knockdown or knockout of the gain-of-function mutant allele is likely necessary in addition to supplementation of the WT BEST1 gene for restoring normal BEST1 activity in these cases. Nevertheless, determining if a patient-derived mutation causes loss or gain of function is essential for designing the treatment strategy.

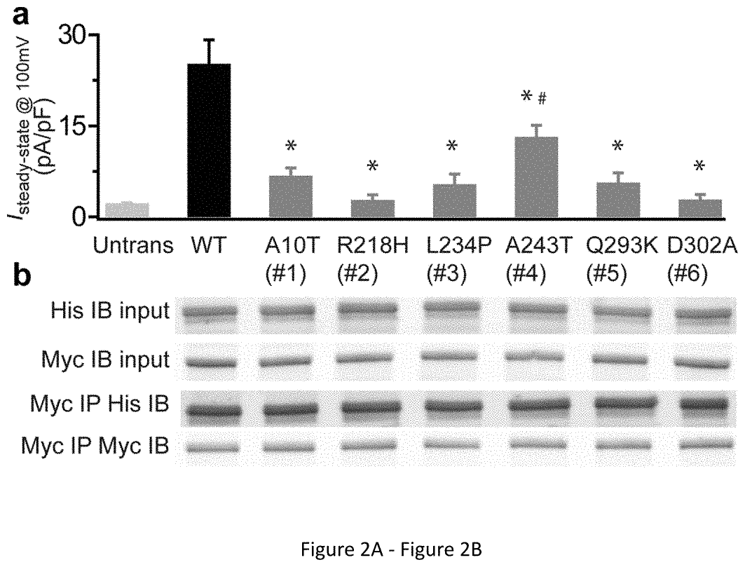

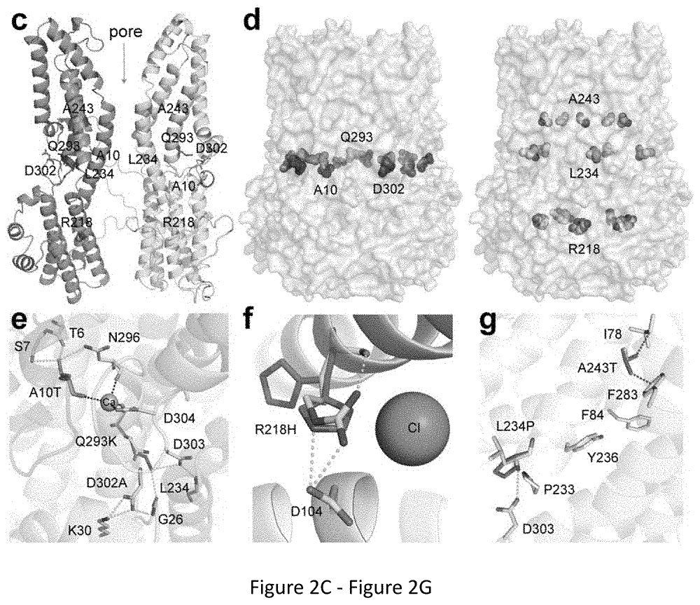

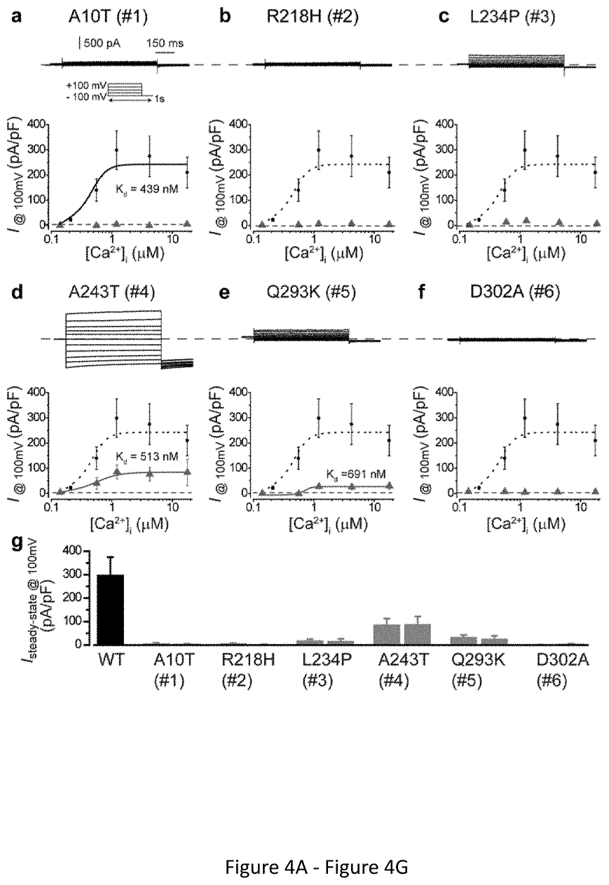

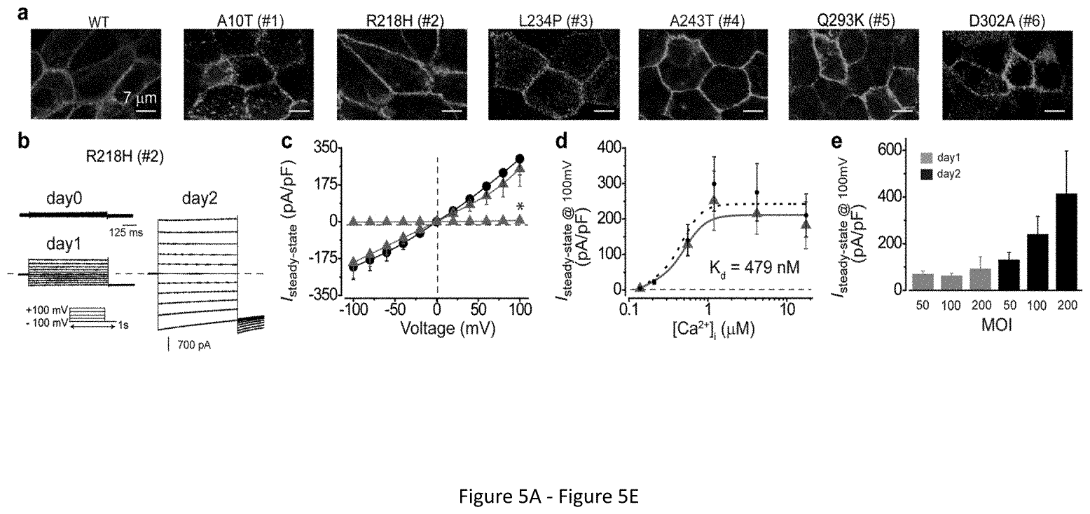

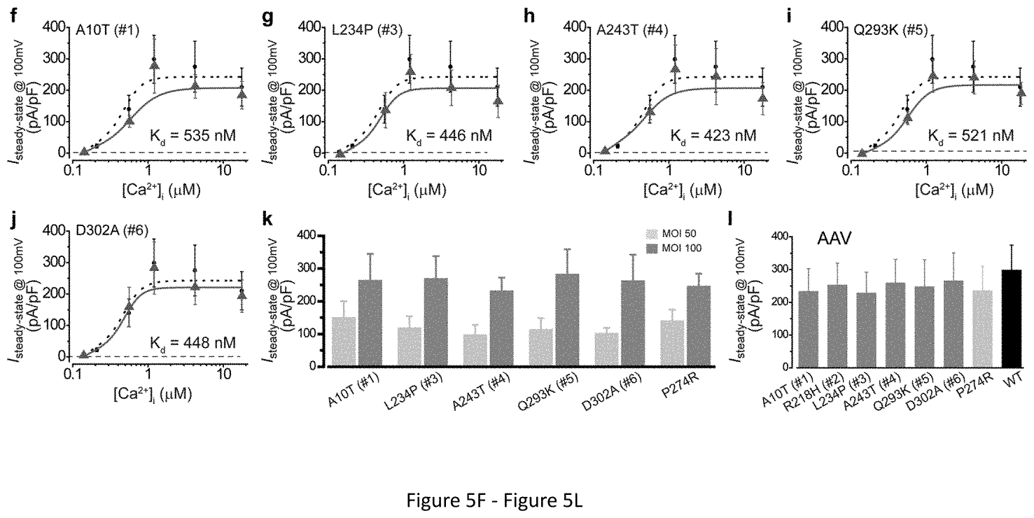

Consistent with the previous report that BEST1 is the channel responsible for Ca²⁺-dependent Cl⁻ currents in RPE (Li, Y. et al., 2017, Elife 6), the channel activity of heterologously expressed BEST1 mutants in HEK293 cells generally reflects the integrity of Ca²⁺-dependent Cl⁻ currents on the plasma membrane of the corresponding patient-derived iPSC-RPEs (FIG. 2 and FIG. 4). Although heterologous expression of WT and mutant channels in HEK293 cells is a standard and powerful approach for functional studies of BEST1, two main limitations have been noticed: firstly, the current density from HEK293 cells transiently transfected with BEST1 is significantly smaller than that from RPE cells; secondly, the Ca²⁺-sensitivity of BEST1-mediated currents in HEK293 cells is left shifted compared to that in RPEs (Li, Y. et al., 2017, Elife 6; Xiao, Q. et al., 2008, J Gen Physiol, 132:681-692). These discrepancies are likely resulted from the intrinsic differences between the two cell types, rather than exogenous vs. endogenous expression of BEST1, as the Ca²⁺-dependent Cl⁻ currents from supplemented WT BEST1 in patient-derived iPSC-RPEs show very similar current density and Ca²⁺-sensitivity as those from endogenous BEST1 in WT iPSC-RPE (FIG. 4 and FIG. 5) (Li, Y. et al., 2017, Elife 6). It is speculated that there are RPE-specific facilitating factor(s) of BEST1.

All three mutations in residues involved in Ca² binding exhibited deficiency in the membrane targeting of BEST1 in iPSC-RPE, suggesting that the membrane trafficking of BEST1 may be facilitated by Ca²⁺ binding. However, previously studies in transiently transfected HEK293 cells showed that mutations around the Ca²⁺-clasp (N296L, E300Q, D301N, D302N, D303L, D304N, E306Q, and N308D) do not affect channel trafficking (Xiao, Q. et al., 2008, J Gen Physiol, 132:681-692). This discrepancy may be attributed to the different cell types and/or mutations in these works.

Further information regarding the data described herein can be found in Ji et al., 2019, Sci Rep 9, 19026, which is incorporated by reference herein in its entirety.

The materials and methods employed in these experiments are now described.

Generation of Human iPSC

Using the CytoTune™-iPS 2.0 Sendai Reprogramming Kit (Thermo Fisher Scientific, A16517), donor-provided skin fibroblasts were reprogrammed into pluripotent stem cells (iPSCs). Immunocytofluorescence assays were carried out following the previously published protocol to score iPSC pluripotency (Li et al., 2016, Methods Mol Biol, 1353:77-88). The iPSCs from all the subjects enrolled in this study were characterized by detecting four standard pluripotency markers (SSEA4, Tra-1-60, SOX2 and Nanog). Nuclei were detected by Hoechst staining. All iPSC lines were passaged every 3-6 days while maintained in mTeSR-1 medium (STEMCELL Technologies, 05850). The morphology and nuclear/cytoplasmic ratio were closely monitored to ensure the stability of the iPSC lines. All the iPSC lines were sent for karyotyping by G-banding to verify genome integrity at Cell Line Genetics (Wisconsin, USA).

Differentiation iPSC into RPE

iPSC lines were cultured to confluence in 6-well culture dishes pretreated with 1:50 diluted matrigel (CORNING, 356230). For the first 14 days, the differentiation medium consisted of Knock-Out (KO) DMEM (Thermo Fisher Scientific, 10829018), 15% KO serum replacement (Thermo Fisher Scientific, 10829028), 2 mM glutamine (Thermo Fisher Scientific, 35050061), 50 U/ml penicillin-streptomycin (Thermo Fisher Scientific, 10378016), 1% nonessential amino acids (Thermo Fisher Scientific, 11140050), and 10 mM nicotinamide (Sigma-Aldrich, N0636). During day 15-28 of differentiation, the differentiation medium was supplemented with 100 ng/ml human Activin-A (PeproTech, 120-14). From day 29 on, the differentiation medium without Activin-A supplementation was used again until differentiation was completed. After roughly 8-10 weeks, dispersed pigmented flat clusters were formatted and manually picked to matrigel-coated dishes. These cells were kept in RPE culture medium as previously described47. It takes another 6-8 weeks in culture for them to form a functional monolayer, which would then be ready for function assays. In addition to well-established classical mature RPE markers (Bestrophin1, CRALBP and RPE65), two more markers (PAX6 and MITF) were also used to validate the RPE fate of the cells. All iPSC-RPE cells in this study were at passage 1. DNA sequencing was used to verify genomic mutations in the mutant iPSC-RPEs.

Cell Lines

HEK293 cells were obtained. As HEK293 is included on the International Cell Line Authentication Committee's list of commonly misidentified cell lines, the cells used in this study were authenticated by short tandem repeat (STR) DNA profiling. The cells were tested negative for mycoplasma contamination, and cultured in DMEM (4.5 g/L glucose, Corning 10013CV) supplemented with 100 μg/ml penicillin-streptomycin and 10% fetal bovine serum.

Electrophysiology

Using an EPC10 patch clamp amplifier (HEKA Electronics) controlled by Patchmaster (HEKA), whole-cell recordings were conducted 24-72 hours after splitting of RPE cells or transfection of HEK293 cells. 1.5 mm thin-walled glass with filament (WPI Instruments) were pulled and fashioned to micropipettes, and filled with internal solution containing (in mM): 130 CsCl, 10 EGTA, 1 MgCl₂, 2 MgATP (added fresh), 10 HEPES (pH 7.4, adjusted by CsOH), and CaCl₂) to obtain the desired free Ca²⁺ concentration (maxchelator.stanford.edu/CaMgATPEGTA-TS.htm). Series resistance was usually 1.5-2.5 MΩ. There was no electronic series resistance compensation. External solution contained (in mM): 140 NaCl, 15 glucose, 5 KCl, 2 CaCl₂), 1 MgCl₂ and 10 HEPES (pH 7.4, adjusted by NaOH). Solution osmolarity was between 310 and 315. A family of step potentials (−100 to +100 mV from a holding potential of 0 mV) were used to generate I-V curves. Currents were sampled at 25 kHz and filtered at 5 or 10 kHz. Traces were acquired at a repetition interval of 4 s (Yang et al., 2014a, Proc Natl Acad Sci USA, 111: 18213-18218). All experiments in this study were carried out at ambient temperature (23±2° C.).

Immunoblot Analysis

Cell pellets were extracted by the M-PER mammalian protein extraction reagent (Thermo Fisher Scientific, 78501) or Mem-PER Plus membrane protein extraction kit (Thermo Fisher Scientific, 89842) with proteinase inhibitors (Roche, 04693159001), and the protein concentration was quantified by a Bio-Rad protein reader. After denaturing at 95° C. for 5 min, the samples (20 μg) were run on 4-15% gradient SDS-PAGE gel at room temperature, and wet transferred onto nitrocellulose membrane at 4° C. The membranes were incubated in blocking buffer containing 5% (w/v) non-fat milk for 1 hour at room temperature, and subsequently incubated overnight at 4° C. in blocking buffer supplemented with primary antibody. Primary antibodies against the following proteins were used for immunoblotting: CRALBP (1:500 Abcam, ab15051), RPE65 (1:1,000 Novus Biologicals, NB100-355), 3-actin (1:2,000 Abcam, ab8227), BESTROPHIN-1 (1:500 Novus Biologicals, NB300-164), His (1:1,000 Fisher Scientific, PA1983B) and Myc (1:1,000 Fisher Scientific, PA1981). Fluorophore-conjugated mouse and rabbit secondary antibodies (LI-COR Biosciences, 925-68070 and 925-32213, respectively) were used at a concentration of 1:10,000 and an incubation time of 1 hour at room temperature, followed by infrared imaging.

Immunoprecipitation

HEK293 cells cultured on 6-cm dishes were co-transfected with pBacman-BEST1(WT)-CFP-myc and pBacman-BEST1(mutant or WT)-YFP-His at 1:1 ratio using PolyJet™ In Vitro DNA Transfection Reagent (SignaGen Laboratories, SL100688) following the manufacturer's standard protocol. 48 h later, cells were harvested by centrifugation at 1000×g for 5 min at room temperature. Cell pellets were lysed in pre-cooled lysis buffer (150 mM NaCl, 50 mM Tris, 0.5% IGEPAL® CA-630, pH 7.4) supplemented with protease inhibitor cocktails (Roche, 04693159001) for 30 min on ice, and then centrifuged at 13,000 rpm for 12 min at 4° C. The supernatants (300 μg) was collected and mixed with 2 μg Myc monoclonal antibody (Thermo Fisher Scientific, MA1-980). After rotating overnight at 4° C., the mixture was incubated with Dynabeads M-280 sheep anti-mouse IgG (Thermo Fisher Scientific, 11202D) for 5 h at 4° C. After thorough washing of the beads, bound fractions were eluted in 1×SDS sample buffer (Biorad, 1610747) by heating for 10 min at 75° C. Proteins were then resolved by SDS-PAGE and analyzed by immunoblotting.

Immunofluorescence

RPE cells cultured on coverslips were washed with PBS twice, and fixed in 4% sucrose and 4% paraformaldehyde at room temperature for 45 min. The fixed cells were permeabilized in PBS containing 0.25% Triton X-100 at room temperature for 10 min. In order to block non-specific binding sites, the samples were incubated with PBS containing 5% BSA at room temperature for 1 h. Primary antibodies were diluted in blocking solution as follows: mouse anti-bestrophin 1 (Novus Biologicals, NB300-164), 1:1000; rabbit anti-collagen IV (Abcam, ab6586), 1:500. The samples were incubated with primary antibody in blocking solution overnight at 4° C. The next day, the samples were washed with PBS thrice. Then, Alexa Fluor 488 conjugated donkey anti-mouse IgG (Thermo Fisher Scientific, A-21202) and Alexa Fluor 647 conjugated donkey anti-rabbit IgG (Thermo Fisher Scientific, A-31573) were diluted in blocking solution and incubated with cells at room temperature for 1 hour. Unbound secondary antibody was washed away with PBS thrice. The samples were then incubated with Hoechst 33342 diluted to 1 μg/ml in PBS at room temperature for 10 min. After thorough washing, coverslips were mounted onto ProLong Diamond Antifade Mountant (Thermo Fisher Scientific, P36966). An Olympus laser scanning confocal microscope was used to acquire images, which were then processed in Fiji. Background was subtracted by the rolling ball method in Fiji with a radius of 50 pixels.

Virus

BacMam baculovirus bearing BEST1-GFP was made in-house as previously described (Goehring et al., 2014, Nature protocols, 9:2574-2585), and added to RPE culture medium at desired MOI (50-200). High titer AAV2 virus (1×10¹² GC/ml) bearing a CMV promoter driven BEST1-T2A-GFP expression cassette was purchased from Applied Biological Materials.

Molecular Cloning

The wild-type BEST1 (synthesized by Genscript), was amplified using polymerase chain reaction (PCR), and was subcloned into a pEGFP-N1 mammalian expression vector. Point mutations BEST1 were made using the In-fusion Cloning Kit (Clontech). All clones were verified by sequencing.

Transfection

20-24 hours before transfection, HEK293 cells were lifted by incubation with 0.25% trypsin at room temperature for 5 min, and split into fresh 3.5-cm culture dishes at proximately 50% confluency. PolyJet transfection reagent (SignaGen SL100688) was utilized to transfect HEK293 cells with plasmids bearing the WT BEST1 or desired mutant (1 μg). 6-8 hours later, the transfection mix was removed, and cells were rinsed with PBS once and cultured in supplemented DMEM. 24 h post transfection, cells were lifted again by trypsin treatment and split onto fibronectin-coated glass coverslips for patch clamp (Yang T. et al., 2013, Nature communications, 4:2540)

Electrophysiological Data and Statistical Analyses

With Patchmaster (HEKA), Microsoft Excel and Origin, patch clamp data were analyzed off-line. Statistical analyses were conducted using built-in functions in Origin. For comparisons between two groups, statistically significant differences between means (P<0.05) were determined using Student's t test. Data are presented as means±s.e.m (Yang et al., 2007, Nat Chem Biol, 3:795-804).

Homology Modeling of Human BEST1

A homology model for BEST1 was generated using the Swiss-Model server from the chicken Best1 crustal structure (Kane Dickson et al., 2014, Nature, 516: 213-218). All figures were made in PyMOL.

Patients and Clinical Analysis

The healthy control donor (WT BEST1) and patients (mutant BEST1) all underwent a complete ophthalmic examination by a retinal physician. This included funduscopy, best-corrected visual acuity, and slit-lamp biomicroscopy. Patients underwent OCT and color fundus photography (Kohl et al., 2015, Nature Genetics, 47: 757-765; McCulloch et al., 2015, Documenta Ophthalmologica Advances in ophthalmology, 130: 1-12). Skin biopsy samples were obtained from the healthy control donor and patients, and processed and cultured as previously described (Li et al., 2016, Methods Mol Biol, 1353, 77-88). For these procedures, patients 2, 4-6 and the parent(s)/legal guardian(s) of patients 1 and 3 provided written informed consent. All methods were performed in accordance with the relevant regulations and guidelines.

The results of the experiments are now described.

Retinal Phenotypes of Six BVMD Patients with Distinct BEST1 Mutations

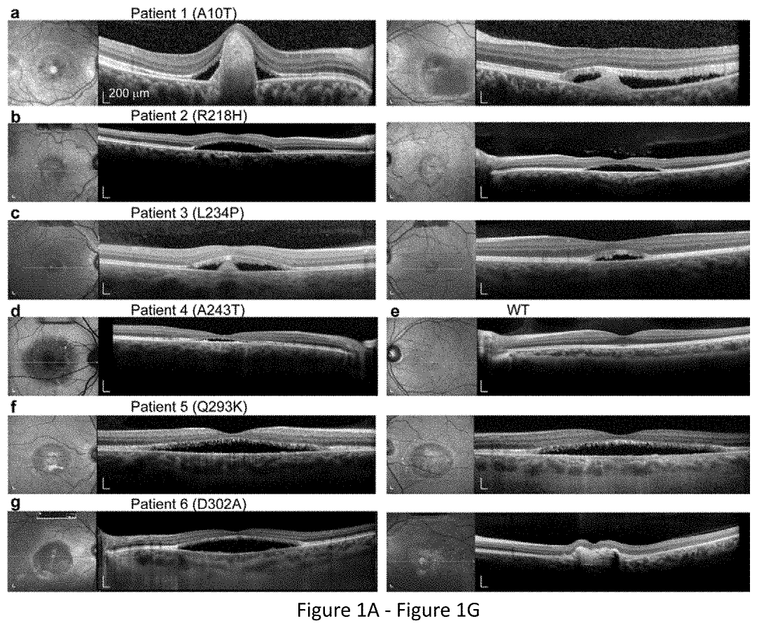

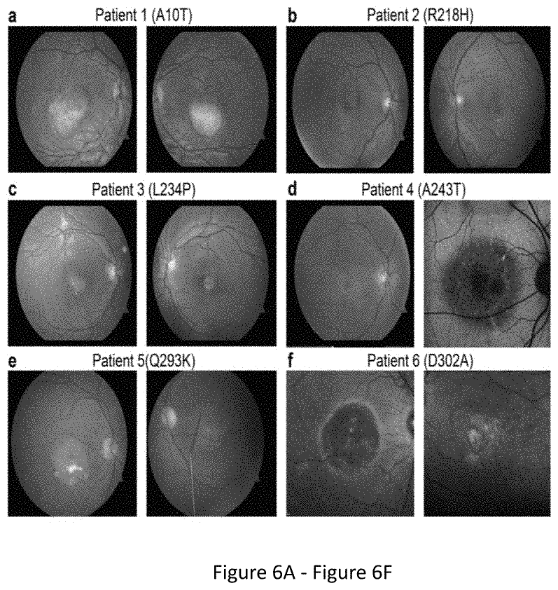

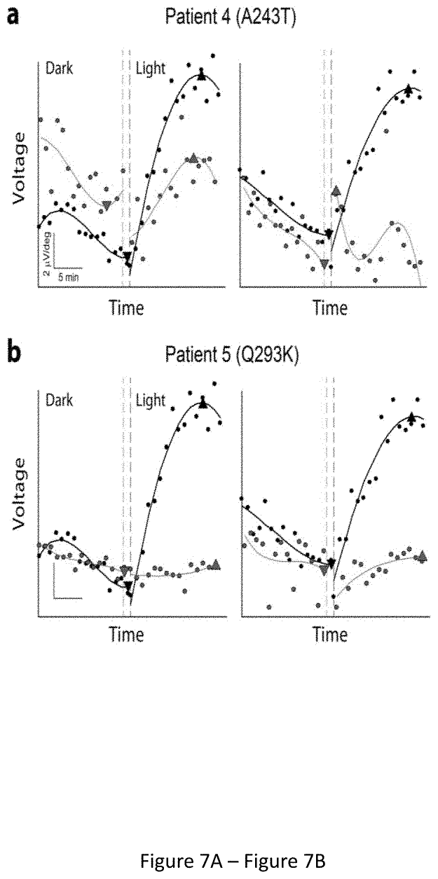

Six diagnosed BVMD patients from unrelated families were studied. Generalized retinal dysfunction was found in all six patients. Fundus autofluorescence imaging and optical coherence tomography (OCT) revealed vitelliform lesions located in the subretinal space, and serous retinal detachments and cystic fluid in the maculae area (FIG. 1 and FIG. 6). Unlike BEST1 recessive patients, whose electroretinography (ERG) and EOG results are significantly different from WT people (Li et al., 2017, Elife, 6: e29914), BVMD patients display normal ERG but abnormal EOG results (FIG. 7).

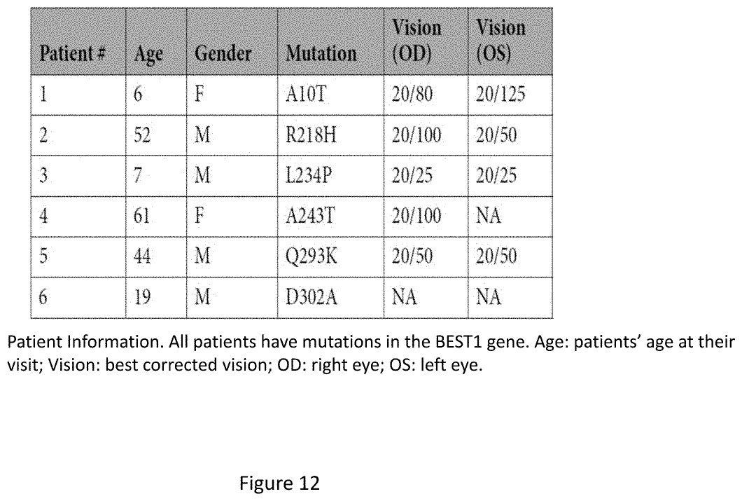

Patient 1, a 6-year-old otherwise healthy girl with a heterozygous c.28G>A; p.A10T mutation, showed reduced visual acuities at 20/80 and 20/125 in the right and left eye, respectively (FIG. 12). Large-area, massive vitelliform lesion was observed in maculae area from both eyes and presented hypo-autofluorescence on fundus autofluorescence imaging. OCT revealed retinal detachments in both eyes with raised fibrotic mounds in the center of the vitelliform lesion and abnormal, elongated photoreceptor outer segments. Intraretinal fluid were also observed from OCT in both eyes (FIG. 1A and FIG. 6A). Patient 2, a 52-year-old otherwise healthy man with a heterozygous c.653G>A; p.R218H mutation, showed reduced visual acuities at 20/100 and 20/50 in the right and left eye, respectively (FIG. 12). OCT detected a thin photoreceptor layer in each eye, extensive subretinal serous fluid and probable vitelliform lesion (FIG. 1B and FIG. 6B). Patient 3, a 7-year-old otherwise healthy boy with a heterozygous c.701T>C; p.L234P mutation, showed reduced visual acuities at 20/25 in both eyes (FIG. 12). Color fundus picture and OCT showed standard boundaries-cleared yellowish vitelliform lesion in macular area of both eyes, as well as subretinal serous fluid and retinal outer segment debris (FIG. 1C and FIG. 6C). Patient 4, a 61-year-old otherwise healthy woman with a heterozygous c.728G>A; p.A243T mutation, showed reduced visual acuities at 20/100 in the right (FIG. 12). No data were recorded for her left eye, which has no light perception due to previous intraocular trauma with a foreign body. The vitelliform material in her right eye displayed hyper-autofluorescence in fundus autofluorescence imaging and was detected in the macular area by OCT (FIG. 1D and FIG. 6D). Her EOG testing consisted of noisy background, and there was a decrease of light rise in both eyes (FIG. 7). Patient 5, a 44-year-old otherwise healthy man with a heterozygous mutation c.877C>A; p.Q293K, showed reduced visual acuities at 20/50 in both eyes (FIG. 12). Hyper-autofluorescence of yellowish subretinal vitelliform deposits were observed in both maculae area (FIG. 1F and FIG. 6E). EOG results showed loss of light rise (FIG. 7). Patient 6 is a 19-year-old otherwise healthy man with a heterozygous c.905A>C; p.D302A mutation (FIG. 12), whose best corrected vision is unknown. Vitelliform lesion with autofluoresence, serous retinal detachments and cystic fluid were found in both maculae area (FIG. 1G and FIG. 6F).

### BEST1 Dominant Mutations Impair the Channel Activity of Best1

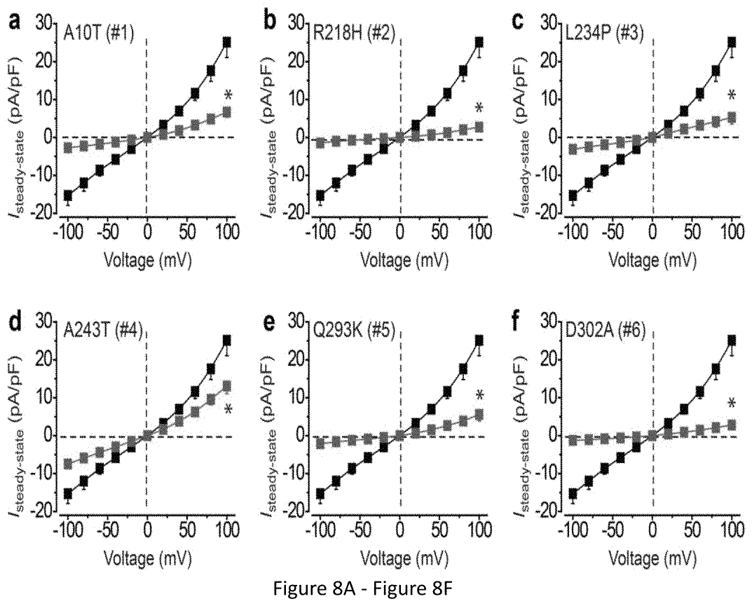

To test the influence of the mutations on BEST1 channel activity, WT and six mutant BEST1 channels were individually introduced into HEK293 cells, which do not have any endogenous Ca²⁺-activated Cl⁻ channel on the plasma membrane (FIG. 2A) (Li, Y. et al., 2017, Elife 6). HEK293 cells expressing BEST1 mutants displayed significantly smaller currents than WT at 1.2 μM [Ca²⁺]ᵢ (FIG. 2A and FIG. 8), where cells expressing WT BEST1 conducted peak current amplitude (Hartzell et al., 2008, Physiol. Rev., 88(2):639-6672). In particular, five mutants (A10T, R218H, L234P, Q293K and D302A) yielded tiny currents with no significant difference from untransfected cells (FIG. 2A and FIG. 8), while the A243T mutant conducted robust currents significantly larger than those from untransfected cells but smaller than those from WT BEST1 (FIG. 2A and FIG. 8). Therefore, these six dominant mutations lead to a complete or partial loss of the BEST1 channel activity.

BEST1 Dominant Mutants Interact with WT

BEST1 channel is a pentamer. To test if the interaction between BEST1 monomers is affected by any of the dominant mutations, mutant BEST1-YFP-His and WT BEST1-CFP-Myc in HEK293 cells were overexpressed, followed by immunoprecipitation with an antibody against Myc and immunoblotting with antibodies against His and Myc, respectively. All six dominant mutants were expressed at similar levels to that of WT BEST1 after transient transfection and retained the interaction with WT BEST1 (FIG. 2B).

### Structural Influence of BEST1 Mutations

To seek the structural bases of the functional results, a BEST1 homology model generated from the structure of chicken bestrophin1 (cBEST1) (FIG. 2C-FIG. 2D) (Li et al., 2017, Elife, 6: e29914; Kane Dickson, et al., 2014, Nature, 516: 213-218; Yang et al., 2014b, Science, 346:355-359; Zhang et al., 2018, Nature communications, 9:3126), which has 74% sequence identity with BEST1, was analyzed. In this model, A10, Q293 and D302 reside in the Ca²⁺-binding sites on the N-terminus or between S4a and S4b (FIG. 2C-FIG. 2D). The A10T and Q293K mutations are predicted to impair the binding of Ca²⁺, which is coordinated by the acidic side chains of the Ca²⁺-clasp and the backbone carbonyl oxygens from A10 and Q293 (FIG. 2E) (Kane Dickson, et al., 2014, Nature, 516: 213-218): the A10T mutation might make additional hydrogen bonds with surrounding residues including N296, one of the Ca²⁺ ligands; the replacement of Q293 with a lysine residue would form new interactions including a hydrogen bond with G26 and an electrostatic interaction with D303 on the Ca²⁺ binding loop. As D303 forms a hydrogen bond with L234 on the transmembrane helix S3b of the adjacent molecule, which contains residues controlling channel gating, the Q293K mutation also seems to have an indirect influence on channel gating. The D302A mutation changes a negative residue to a hydrophobic residue in the carboxylate loop, potentially weakening the binding of Ca²⁺ to the channel (FIG. 2E). Moreover, this mutation may destabilize the Ca²⁺ binding loop since it presumably eliminates a hydrogen bond with G26 and an electrostatic interaction with K30. Therefore, the A10T, Q293K and D302A mutations may prohibit channel activation by diminishing Ca²⁺ binding, which is absolutely required for BEST1 to conduct current (Li et al., 2017, Elife, 6: e29914; Sun et al., 2002, Proc Natl Acad Sci USA, 99: 4008-4013).

R218 is localized on the alpha helix S3a (FIG. 2C and FIG. 2D), which falls on a putative Cl-binding site in the channel inner cavity (Kane Dickson et al., 2014, Nature, 516:213-218). So the R218H mutation may decrease the local concentration of anions at the permeation pore, thereby disrupting channel activity. The model structure of the R218H mutant also predicts more flexibility of H218 compared to R218 because of histidine's smaller side chain. Furthermore, H218 lacks a hydrogen bond with its own carbonyl O atom and is presumably located farther from D104 on the adjacent molecule compared to R218. Hence, the R218H mutant might destabilize the local structure and weaken the interaction between monomers.

L234 and A243 are localized on the transmembrane alpha helix S3b (FIG. 2C and FIG. 2D), which contains multiple residues (e.g. P233 and Y236) critical for channel gating (FIG. 2G) (Ji et al., Commun Biol, 2: 240; Miller et al., 2019, Elife, 8:e43231). In fact, the model structures predict that the L234P mutation cannot form a hydrogen bond with D303 from the adjacent molecule, while the A243T mutation may have steric hindrances with 178 in the same molecule and F283 from the adjacent molecule (FIG. 2G). Moreover, the L234P mutant may have a highly flexible structure around the mutation site due to consecutive proline residues.

### Mutations in Residues Involved in Ca²⁺ Binding Disrupt Membrane Localization of BEST1

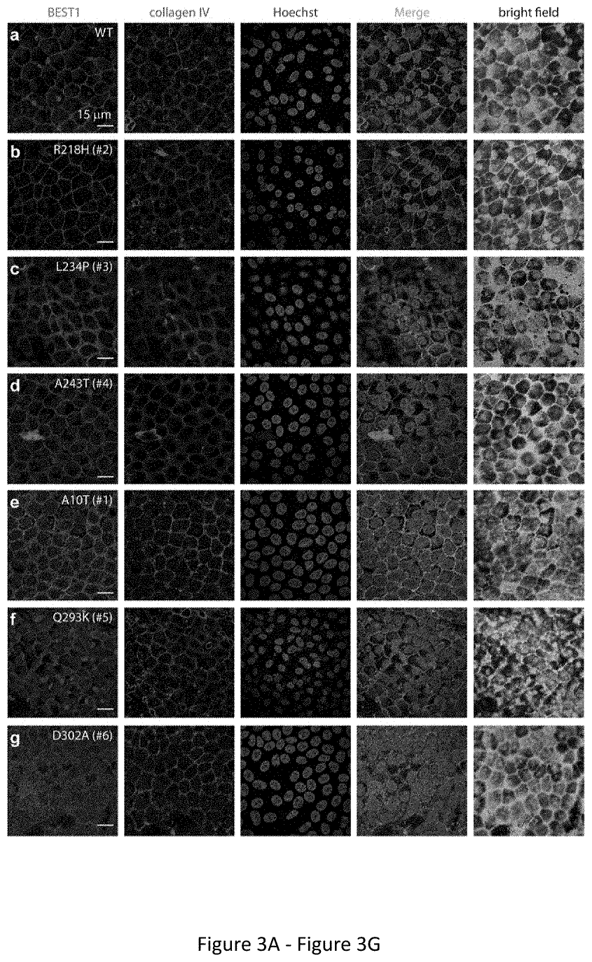

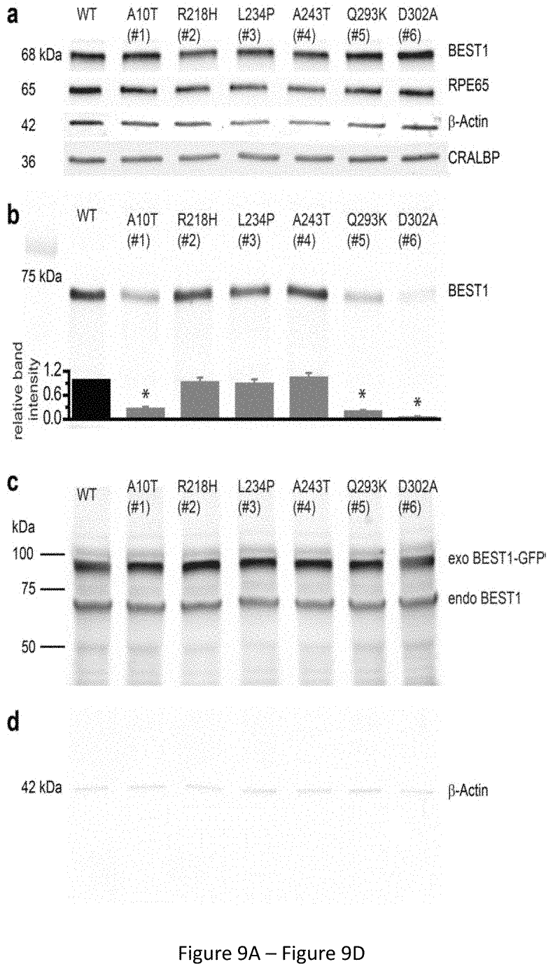

To directly examine the physiological impact of the six patient-specific BEST1 dominant mutations, induced pluripotent stem cells (iPSCs) were reprogrammed from the patients' skin cells and then differentiated to RPE cells (iPSC-RPEs) (Kittredge et al, 2018, Journal of visualized experiments, 138: e57791). The RPE status of the cells was confirmed by morphological signatures including intracellular pigment and hexagonal shape (FIG. 3). RPE-specific marker proteins RPE65 (retinal pigment epithelium-specific 65 kDa protein) and CRALBP (cellular retinaldehyde-binding protein) were well expressed in iPSC-RPEs derived from a BEST1 WT donor and the patients as shown by immunoblotting (FIG. 9), confirming the mature status of all iPSC-RPEs. Moreover, all six patient-derived iPSC-RPEs showed a similar overall BEST1 expression level compared to that in iPSC-RPE derived from the BEST1 WT donor (FIG. 9), indicating that none of the six mutations impairs the protein expression of the channel.

The subcellular localization of BEST1 in iPSC-RPEs was then examined by immunostaining. R218H, L234P and A243T displayed normal BEST1 signal on the plasma membrane just like the WT (FIG. 3A through FIG. 3D). By contrast, all three mutations in residues involved in Ca²⁺ binding exhibited deficiency in the membrane targeting of BEST1: D302A has the strongest phenotype with a complete loss of BEST1 antibody staining signal on the plasma membrane, while A10T and Q293K both partially lost membrane localization of BEST1 (FIG. 3E through FIG. 3G). Consistently, immunoblotting showed decreased levels of the A10T, Q293K and D302A mutant proteins on the cell membrane (FIG. 9).

Deficient Ca²⁺-Dependent Cl⁻ Current in iPSC-RPEs Bearing BEST1 Dominant Mutations

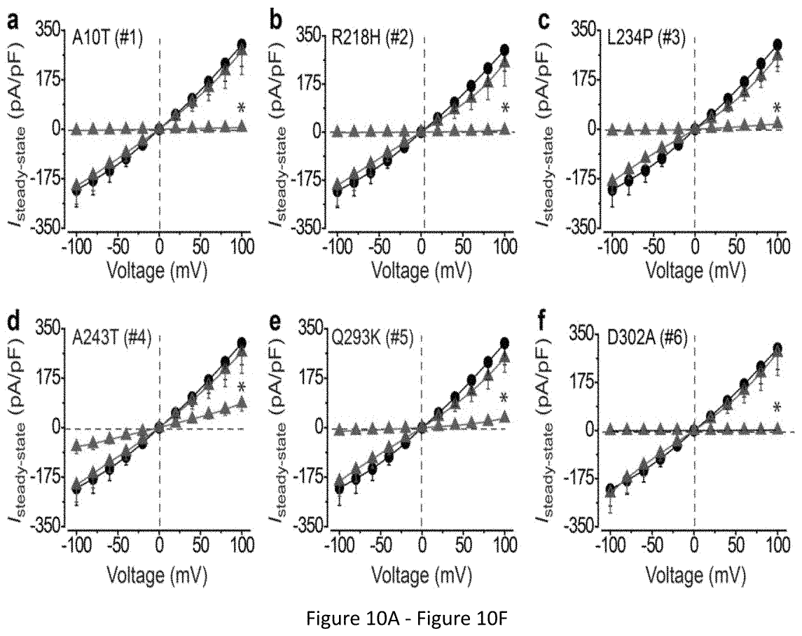

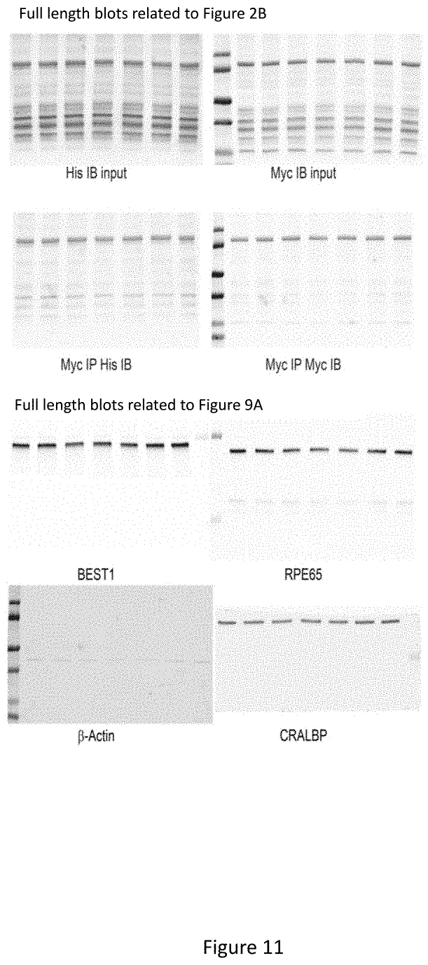

To elucidate the influences of the mutations on the physiological activity of BEST1, Ca²⁺-dependent Cl⁻ current was measured in the patient-derived iPSC-RPEs by whole-cell patch clamp (FIG. 4A through FIG. 4F and FIG. 10). Remarkably, tiny currents (<6 pA/pF) were detected in the A10T, R218H and D302A patient-derived iPSC-RPEs at all tested [Ca²⁺]ᵢ (FIG. 4A, FIG. 4B, FIG. 4F, FIG. 10), suggesting a complete loss of BEST1 channel activity in those mutants. On the other hand, robust currents were detected in the L234P, A243T and Q293K patient-derived iPSC-RPE, but the current amplitude was significantly reduced compared to that from iPSC-RPE with WT BEST1 (FIG. 4C through FIG. 4E and FIG. 10), suggesting a partial loss of function. Moreover, as currents from the A243T and Q293K iPSC-RPEs were large enough for fitting to the Hill equation, their Ca²⁺-sensitivity was calculated: compared to that from the WT iPSC-RPE (K_{d}=439 nM), Ca²⁺-sensitivity was normal in A243T iPSC-RPE (K_{d}=513 nM) but significantly right shifted in Q293K iPSC-RPE (K_{d}=691 nM, FIG. 4D-FIG. 4E), consistent with the structure model in which Q293 but not A243 is involved in Ca²⁺-binding (FIG. 2C). L234P is not expected to affect Ca²⁺-sensitivity, because L234 is localized outside of the Ca²⁺-clasp (FIG. 2C). For each mutation, similar electrophysiological results were obtained from two clonal iPSC-RPEs (FIG. 4G), indicating that the observed defect in Ca²⁺-dependent Cl⁻ current is mutation-specific.

Taken together, these results showed that the six mutations analyzed in this work can be classified into three different groups by the phenotypes: complete loss of function (A10T, R218H and D302A), and partial loss of function with normal (A243T and L234P) or decreased (Q293K) Ca²⁺-sensitivity.

### Rescue of BEST1 Dominant Mutations by Gene Supplementation

It was previously reported that the defective Ca²⁺-dependent Cl⁻ current in patient-derived iPSC-RPEs carrying recessive BEST1 mutations can be rescued by baculovirus (BV)-mediated supplementation of the WT BEST1 gene (Li et al., 2017, Elife, 6: e29914). To investigate if the Ca²⁺-dependent Cl⁻ current is rescuable in iPSC-RPEs bearing BEST1 dominant mutations, WT BEST1-GFP was expressed from a BV vector in the six patient-derived BEST1 iPSC-RPEs. Confocal imaging confirmed that WT BEST1-GFP is localized on the plasma membrane of all six patient-derived iPSC-RPEs (FIG. 5A), including A10T, Q293K and D302A iPSC-RPEs in which the membrane localization of endogenous BEST1 is impaired to different degrees (FIG. 3).

For electrophysiological analysis, first iPSC-RPE carrying the BEST1 R218H mutation was utilized to optimize the time course and MOI of virus infection, as R218H is a null mutation with normal membrane localization of endogenous BEST1, representing a “clean” case with strong phenotypes. Ca²⁺-dependent Cl⁻ current measured at 1.2 μM [Ca²⁺]ᵢ by whole-cell patch clamp significantly increased from 24 to 48-hours, and in a dose-dependent manner at 48-hours post infection (FIG. 5B-FIG. 5E). A complete rescue of the Cl⁻ current at peak [Ca²⁺]ᵢ was observed at 48-hours post infection with a minimum MOI of 100 (FIG. 5C-FIG. 5E and FIG. 10), where Ca²⁺-dependent Cl⁻ currents in a full range of [Ca²⁺]ᵢs were also fully restored (FIG. 5D). Consistently, Ca²⁺-dependent Cl⁻ currents in the other five patient-derived iPSC-RPEs were all rescued to a similar level under the same conditions (FIG. 5F through FIG. 5K), regardless of the type or level of deficiency in the endogenous BEST1 function. Immunoblotting results showed that the exogenous BEST1 expression level is comparable to that of the endogenous BEST1 (FIG. 9).

Moreover, the efficacy of rescue in iPSC-RPEs bearing BEST1 dominant mutations was comparable to that in a previously reported iPSC-RPE with a recessive P274R mutation (FIG. 5K) (Li et al., 2017, Elife, 6: e29914). Taken together, it is concluded that the defect of Ca²⁺-dependent Cl⁻ conductance caused by a BEST1 loss-of-function mutation, either dominant or recessive, is rescuable by BV-mediated supplementation of the WT BEST1 gene with the same dosage and time course.

To test if BEST1 supplementation can be mediated by adeno-associated virus (AAV), which has been approved for gene therapy in the human retina (Russel et al., 2017, Lancet, 390(10097):849-860), iPSC-RPEs was infected with an AAV serotype 2 (AAV2) viral vector expressing BEST1-T2A-GFP. Consistent with the results from BV-mediated supplementation, Ca²⁺-dependent Cl⁻ currents were restored in iPSC-RPEs bearing either a dominant or recessive BEST1 mutation (FIG. 5L), providing a proof-of-concept for curing BEST1-associated retinal degenerative diseases in both dominant and recessive cases by AAV-mediated gene augmentation.

### Influence of BEST1 Dominant Mutations on BEST1 Channel Function

Here studies are presented that comprehensively examined six BEST1 dominant disease-causing mutations (A10T, R218H, L234P, A243T, Q293K and D302A) derived from BVMD patients in an interdisciplinary platform, including whole-cell patch clamp with patient-derived iPSC-RPEs and HEK293 cells expressing the mutant channels, immunodetection of endogenous BEST1 in iPSC-RPEs, structural analyses with human homology models, and virus-mediated BEST1 gene supplementation. Collectively, these results illustrate the physiological influence of these six dominant mutations on RPE surface Ca²⁺-dependent Cl⁻ current and the BEST1 channel function, provide structural insights into their disease-causing mechanisms, and demonstrate the rescue of BEST1 function in iPSC-RPE via gene supplementation. Notably, the diminished Ca²⁺-dependent Cl⁻ currents in the R218H, L234P and A243T patient-derived iPSC-RPEs are in accord with the deficient Ca²⁺-stimulated Cl⁻ secretion shown by Cl⁻ sensitive fluorescent dyes in these cells (Moshfegh et al., 2016, Human molecular genetics, 25: 2672-2680.

Previously, it was reported that the impaired Ca²⁺-dependent Cl⁻ current in iPSC-RPE derived from an ARB patient bearing a BEST1 recessive mutation (P274R) was rescuable by BV-mediated supplementation of WT BEST1 (Li et al., 2017, Elife, 6: e29914). Here, it is shown that the same strategy, with both BV and AAV2, can be generally applied to restore Ca²⁺-dependent Cl⁻ current impaired by BEST1 loss-of-function dominant mutations, which can be sub-classified into null (e.g. A10T, R218H and D302A), and partial deficiency with unaffected (e.g. L234P and A243T) or shifted (e.g. Q293K) Ca²⁺-sensitivity.

The retina has been the frontier of translational gene therapy in the past 20 years. Recently, the first gene therapy drug, an AAV-based vector carrying a correct copy of the RPE65 gene, was approved by FDA for treating retinal degenerative Leber congenital amaurosis type 2 (LCA2), which is caused by recessive mutations in RPE65 (Russell et al., 2017, Lancet, 390: 849-860; Jacobson et al., 2012, Arch Ophthalmol, 130: 9-24; Testa et al., 2013, Ophthalmology, 120: 1283-1291; Bainbridge et al., 2008, The New England journal of medicine, 358: 2231-2239; Bennet et al., 2016, Lancet, 661-672) As another inherited retinal disorder clearly linked to the mutation of a single gene, bestrophinopathy represents an attractive target of gene therapy. However, since the vast majority of known BEST1 mutations are autosomal dominant, it remains a critical question whether the dominant mutant allele should be purposely suppressed during therapeutic intervention. The results showed that virus-mediated WT BEST1 gene supplementation restores the diminished Ca²⁺-dependent Cl⁻ currents in patient-derived iPSC-RPEs with the same dose- and time-dependent efficacy regardless of the mutation type (dominant vs. recessive) or deficiency level (null vs. partial), providing one of the first lines of evidence that BEST1 dominant mutations are rescuable by WT gene augmentation without the need of disrupting/suppressing the mutant allele. In agreement with these findings, a preprint by Sinha et al. showed that two more BEST1 dominant mutations, namely R218C and N296H, can be rescued by lentivirus-mediated gene augmentation in iPSC-RPE cells (Sinha et al., 2019, bioRxiv, 796581). Interestingly, a third dominant mutation in that report, A164K, was not responsive to gene augmentation, probably attributed to structural instability as suggested by the authors (Sinha et al., 2019, bioRxiv, 796581).

Several gain-of-function mutations (e.g. D203A, I205T and Y236C) were recently identified which significantly enhance BEST1 channel activity (Ji et al., 2019, Commun Biol, 2: 240). Mechanistically, these mutations dysregulate BEST1 gating at two Ca²⁺-dependent gates, resulting in increased channel opening (Ji et al., 2019, Commun Biol, 2: 240). Although the pathological basis of elevated BEST1 activity remains unclear, it is speculated that knockdown or knockout of the gain-of-function mutant allele is likely necessary in addition to supplementation of the WT BEST1 gene for restoring normal BEST1 activity in these cases. However, due to the unavailability of patient-derived RPEs bearing gain-of-function mutations, whether the endogenous BEST1 protein level is negatively affected by these mutations remains unclear. Nevertheless, determining whether a patient-derived mutation causes a loss or gain of function is essential for designing the treatment strategy.

Consistent with the previous report that BEST1 is the channel responsible for Ca²⁺-dependent Cl⁻ currents in RPE (Li et al., 2017, Elife, 6: e29914), the channel activity of heterologously expressed BEST1 mutants in HEK293 cells generally reflects the integrity of Ca²⁺-dependent Cl⁻ currents on the plasma membrane of the corresponding patient-derived iPSC-RPEs (FIG. 2 and FIG. 4).

All three mutations in residues predicted to be involved in Ca²⁺ binding exhibited deficiency in the membrane targeting of endogenous BEST1 in iPSC-RPE, suggesting that the membrane trafficking of BEST1 may be facilitated by Ca²⁺ binding. However, previous studies in transiently transfected HEK293 cells showed that mutations around the Ca²⁺-clasp (N296L, E300Q, D301N, D302N, D303L, D304N, E306Q, and N308D) do not affect channel trafficking (Xiao et al., 2008, J Gen Physiol, 132: 681-692). This discrepancy may be attributed to the different cell types and/or mutations in these works.

In summary, the experiments described herein examined the clinical, electrophysiological and structural impacts of six BEST1 dominant mutations, and demonstrated the restoration of BEST1 function in iPSC-RPEs bearing dominant mutations by virus-mediated gene augmentation. Importantly, gene augmentation therapy also has great potential to treat other inherited disorders in the retina, such as autosomal dominant retinitis pigmentosa (adRP), which can be caused by mutations in over 25 known genes including RHO and RPE65 (Daiger et al., 2015, Cold Spring Harbor perspectives in medicine, 5: a017129) RHO is the most frequently mutated gene associated with adRP. AAV-mediated RHO augmentation partially rescues retinal degeneration in the well-characterized R23H transgenic mouse model (Lewin et al., 2014, Cold Spring Harbor perspectives in medicine, 4: a017400), which exhibits loss-of-function evidenced by reduced rhodopsin levels (Wu et al., 1998, Neuroscience, 87: 709-717; Noorwez et al., 2009, The Journal of biological chemistry, 284: 33333-33342; Kemp et al., 1992, Am J Ophthalmol, 113, 165-174). On the other hand, while RPE65 is mainly associated with LCA, a D477G mutation in it has been linked to adRP (Bowne et al., 2011, Eur J Hum Genet, 19: 1074-1081). Heterozygous RPE65 D477G knock-in mice exhibited reduced isomerase activity and delayed dark adaptation (Shin et al., 2017, The American journal of pathology, 187: 517-527), suggesting a loss-of-function phenotype. Therefore, these results raise the possibility of curing adRP associated with RPE65 by the FDA approved AAV-RPE65 vector without suppressing the dominant D477G mutant allele.

### Example 2: Sequences

(wildtype BEST1 amino acid sequence)
SEQ ID NO: 1
MTITYTSQVANARLGSFSRLLLCWRGSIYKLLYGEFLIFLLCYYBRFIYRLALTEE
QQUVIFEKLTLYCDSYIQLIPISFVLGFYVTLVVTRWWNQYENLPWPDRLMSLVS
GFVEGKDEQGRLLRRTLIRYANLGNVLILRSVSTAVYKREPSAQHLVQAGFMTP
AEHKQLEKLSLPHNMFWVPWVWFANLSMKAWLGGRIRDPILLQSLLNEMNTLR
TQCGHLYAYDWISIPLVYTQVVTVAVYSFFLTCLVGRQFLNPAKAYPGHELDLV
VPVETFLQFFFYVGWLKVAEQLINPFGEDDDDFETNWIVDRNLQVSLLAVDEMH
QDLPRMEPDMYWNKPEPQPPYTAASAQFRRASFMGSTENISLNKEEMEFQPNQE
DEEDAHAGIIGRFLGLQSHDHHPPRANSRTKLLWPKRESLLHEGLPKNHKAAKQ
NVRGQEDNKAWKLKAVDAFKSAPLYQRPGYYSAPQTPLSPTPMFFPLEPSAPSK
LHSVTGIDTKDKSLKTVSSGAKKSFELLSESDGALMEHPEVSQVRRKTVEFNLTD
MPEIPENHLKEPLEQSPTNIHTTLKDHMDPYWALENRDEAHS
(wildtype BEST1 nucleic acid sequence)
SEQ ID NO: 2
ATGACCATCACTTACACAAGCCAAGTGGCTAATGCCCGCTTAGGCTCCTTCTC
CCGCCTGCTGCTGTGCTGGCGGGGCAGCATCTACAAGCTGCTATATGGCGAG
TTCTTAATCTTCCTGCTCTGCTACTACATCATCCGCTTTATTTATAGGCTGGCC
CTCACGGAAGAACAACAGCTGATGTTTGAGAAACTGACTCTGTATTGCGACA
GCTACATCCAGCTCATCCCCATTTCCTTCGTGCTGGGCTTCTACGTGACGCTG
GTCGTGACCCGCTGGTGGAACCAGTACGAGAACCTGCCGTGGCCCGACCGCC
TCATGAGCCTGGTGTCGGGCTTCGTCGAAGGCAAGGACGAGCAAGGCCGGCT
GCTGCGGCGCACGCTCATCCGCTACGCCAACCTGGGCAACGTGCTCATCCTG
CGCAGCGTCAGCACCGCAGTCTACAAGCGCTTCCCCAGCGCCCAGCACCTGG
TGCAAGCAGGCTTTATGACTCCGGCAGAACACAAGCAGTTGGAGAAACTGAG
CCTACCACACAACATGTTCTGGGTGCCCTGGGTGTGGTTTGCCAACCTGTCAA
TGAAGGCGTGGCTTGGAGGTCGAATCCGGGACCCTATCCTGCTCCAGAGCCT
GCTGAACGAGATGAACACCTTGCGTACTCAGTGTGGACACCTGTATGCCTAC
GACTGGATTAGTATCCCACTGGTGTATACACAGGTGGTGACTGTGGCGGTGT
ACAGCTTCTTCCTGACTTGTCTAGTTGGGCGGCAGTTTCTGAACCCAGCCAAG
GCCTACCCTGGCCATGAGCTGGACCTCGTTGTGCCCGTCTTCACGTTCCTGCA
GTTCTTCTTCTATGTTGGCTGGCTGAAGGTGGCAGAGCAGCTCATCAACCCCT
TTGGAGAGGATGATGATGATTTTGAGACCAACTGGATTGTCGACAGGAATTT
GCAGGTGTCCCTGTTGGCTGTGGATGAGATGCACCAGGACCTGCCTCGGATG
GAGCCGGACATGTACTGGAATAAGCCCGAGCCACAGCCCCCCTACACAGCTG
CTTCCGCCCAGTTCCGTCGAGCCTCCTTTATGGGCTCCACCTTCAACATCAGC
CTGAACAAAGAGGAGATGGAGTTCCAGCCCAATCAGGAGGACGAGGAGGAT
GCTCACGCTGGCATCATTGGCCGCTTCCTAGGCCTGCAGTCCCATGATCACCA
TCCTCCCAGGGCAAACTCAAGGACCAAACTACTGTGGCCCAAGAGGGAATCC
CTTCTCCACGAGGGCCTGCCCAAAAACCACAAGGCAGCCAAACAGAACGTTA
GGGGCCAGGAAGACAACAAGGCCTGGAAGCTTAAGGCTGTGGACGCCTTCA
AGTCTGCCCCACTGTATCAGAGGCCAGGCTACTACAGTGCCCCACAGACGCC
CCTCAGCCCCACTCCCATGTTCTTCCCCCTAGAACCATCAGCGCCGTCAAAGC
TTCACAGTGTCACAGGCATAGACACCAAAGACAAAAGCTTAAAGACTGTGAG
TTCTGGGGCCAAGAAAAGTTTTGAATTGCTCTCAGAGAGCGATGGGGCCTTG
ATGGAGCACCCAGAAGTATCTCAAGTGAGGAGGAAAACTGTGGAGTTTAACC
TGACGGATATGCCAGAGATCCCCGAAAATCACCTCAAAGAACCTTTGGAACA
ATCACCAACCAACATACACACTACACTCAAAGATCACATGGATCCTTATTGG
GCCTTGGAAAACAGGGATGAAGCACATTCC

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating a retinal degenerative disorder associated with a BEST1 dominant mutation in a subject, the method comprising administering to a subject in need thereof an effective amount of a composition comprising a nucleic acid molecule encoding wild-type BEST1.

2. The method of claim 1, wherein the nucleic acid molecule encodes a polypeptide comprising an amino acid sequence comprising SEQ ID NO: 1.

3. The method of claim 1, wherein the nucleic acid molecule comprises a nucleic acid sequence selected from the group consisting of SEQ ID NO: 2 and a nucleic acid sequence that is at least 90% homologous to SEQ ID NO: 2.

4. The method of claim 1, wherein the composition comprises a recombinant AAV promoter linked to the nucleic acid.

5. The method of claim 4, wherein the recombinant AAV promoter is an AAV2 promoter.

6. The method of claim 1, wherein the composition comprises a recombinant AAV vector encoding BEST1.

7. The method of claim 6, wherein the recombinant AAV vector is an AAV2 vector.

8. The method of claim 1, wherein the dominant mutation is selected from the group consisting of p.A10T, p.R218H, p.L234P, p.A243T, p.Q293K and p.D302A.

9. The method of claim 1, wherein the composition is administered via subretinal injection.

10. The method of claim 1, wherein the composition is administered to a retinal pigment epithelial cells of the subject.

11. The method of claim 1, wherein the retinal degenerative disorder is a bestrophinopathy selected from the group consisting of: Best vitelliform macular dystrophy (BVMD), adult-onset vitelliform dystrophy (AVMD), autosomal dominant vitreoretinochoroidopathy (ADVIRC), and retinitis pigmentosa (RP).

12. The method of claim 1, wherein the subject is a mammal.

13. The method of claim 12, wherein the mammal is a human.

14. A cell having an endogenous BEST1 dominant mutation comprising an exogenous nucleic acid molecule that encodes wild-type BEST1.

15. The cell of claim 14, wherein the exogenous nucleic acid molecule encodes polypeptide comprising an amino acid sequence comprising SEQ ID NO: 1.

16. The cell of claim 15, wherein the exogenous nucleic acid molecule comprises a nucleic acid sequence selected from the group consisting of SEQ ID NO: 2 and a nucleic acid sequence that is at least 90% homologous to SEQ ID NO: 2.

17. The cell of claim 14, wherein the exogenous nucleic acid molecule comprises a recombinant AAV promoter linked to the wild-type BEST1.

18. The cell of claim 17, wherein the recombinant AAV promoter is an AAV2 vector.

19. The cell of claim 14, wherein the exogenous nucleic acid molecule comprises a recombinant AAV vector encoding BEST1.

20. The cell of claim 19, wherein the recombinant AAV vector is an AAV2 vector.

21. The cell of claim 14, wherein the cell is retinal pigment epithelial cell.


SEQUENCE LISTING (2 sequences)

SEQ ID NO: 1 - protein, 585 aa, Homo sapiens
MTITYTSQVANARLGSFSRLLLCWRGSIYKLLYGEFLIFLLCYYIIRFIYRLALTEEQQLMFEKLTLYCDSYIQLIPISFVLGFYVTLVVTRWWNQYENLPWPDRLMSLVSGFVEGKDEQGRLLRRTLIRYANLGNVLILRSVSTAVYKRFPSAQHLVQAGFMTPAEHKQLEKLSLPHNMFWVPWVWFANLSMKAWLGGRIRDPILLQSLLNEMNTLRTQCGHLYAYDWISIPLVYTQVVTVAVYSFFLTCLVGRQFLNPAKAYPGHELDLVVPVFTFLQFFFYVGWLKVAEQLINPFGEDDDDFETNWIVDRNLQVSLLAVDEMHQDLPRMEPDMYWNKPEPQPPYTAASAQFRRASFMGSTFNISLNKEEMEFQPNQEDEEDAHAGIIGRFLGLQSHDHHPPRANSRTKLLWPKRESLLHEGLPKNHKAAKQNVRGQEDNKAWKLKAVDAFKSAPLYQRPGYYSAPQTPLSPTPMFFPLEPSAPSKLHSVTGIDTKDKSLKTVSSGAKKSFELLSESDGALMEHPEVSQVRRKTVEFNLTDMPEIPENHLKEPLEQSPTNIHTTLKDHMDPYWALENRDEAHS

SEQ ID NO: 2 - DNA, 1755 bp, Homo sapiens
atgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggctggccctcacggaagaacaacagctgatgtttgagaaactgactctgtattgcgacagctacatccagctcatccccatttccttcgtgctgggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgagatgaacaccttgcgtactcagtgtggacacctgtatgcctacgactggattagtatcccactggtgtatacacaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtggcagagcagctcatcaacccctttggagaggatgatgatgattttgagaccaactggattgtcgacaggaatttgcaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctgccccactgtatcagaggccaggctactacagtgccccacagacgcccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacagggatgaagcacattcc